(19) United States
(12) Patent Application Publication
(10) Pub. No.: US 2018/0289949 A1
(43) Pub. Date: Oct. 11, 2018

(54) Neural Probe Systems, Methods, And Devices

(71) Applicant: NeuroOne, Inc., Minneapolis, MN (US)

(72) Inventors: Thomas Bachinski, Lakeville, MN (US); Wade Fredrickson, Shorewood, MN (US); Mark Christianson, Eden Prairie, MN (US)

(21) Appl. No.: 15/942,929

(22) Filed: Apr. 2, 2018

Related U.S. Application Data
(60) Provisional application No. 62/480,159, filed on Mar. 31, 2017.
(60) Provisional application No. 62/577,394, filed on Oct. 26, 2017.

(51) Int. Cl. A61N 1/05; A61B 5/00; A61B 18/14

(52) U.S. Cl. CPC A61N 1/0529; A61B 5/04001; A61B 18/1492; A61B 5/6868

(57) ABSTRACT

Provided herein are improved neural probes for detection and stimulation, including improved depth electrodes and cortical electrodes, along with various related improved components, devices, methods, and technologies.

### CROSS-REFERENCE TO RELATED APPLICATION(S)

This application claims the benefit under 35 U.S.C. § 119(e) to U.S. Provisional Application 62/480,159, filed Mar. 31, 2017 and entitled “Neural Probe Systems, Methods, and Devices,” and to U.S. Provisional Application 62/577,394, filed Oct. 26, 2017 and entitled “Neural Probe Systems, Methods, and Devices,” both of which are hereby incorporated herein by reference in their entireties.

### FIELD OF THE INVENTION

The various embodiments herein relate to neural probes, including electrode arrays, and related systems and methods for detection and/or stimulation.

### BACKGROUND OF THE INVENTION

The various embodiments herein relate to neural probes, including neural detection, stimulation, and ablation probes and devices, and further including related components, devices, and technologies. Known neural probes and devices have relatively thick profiles that can result in damage to the patient's brain tissue during use. Further, the known devices and technologies are constructed in a scale that is larger than the molecular level, which reduces flexibility and efficiency in construction and modification. In addition, many known devices are too large or are otherwise configured such that they cannot be used in minimally invasive procedures.

There is a need in the art for improved neural probes and related devices and technologies.

### BRIEF SUMMARY OF THE INVENTION

Discussed herein are various neural probes in the form of electrodes and other related devices, methods, and technologies.

In Example 1, a depth electrode comprises an elongate, unitary tubular body, at least one lumen defined through a length of the elongate tubular body, and at least one electrode array disposed on an outer surface of the elongate tubular body.

Example 2 relates to the depth electrode according to Example 1, wherein the at least one electrode array comprises a thin conductive film comprising a plurality of electrode contacts.

Example 3 relates to the depth electrode according to Example 1, wherein the at least one electrode array is disposed around the elongate tubular body in a spiral configuration.

Example 4 relates to the depth electrode according to Example 1, wherein the at least one electrode array comprises at least two elongate electrode arrays disposed along a length of the elongate tubular body.

Example 5 relates to the depth electrode according to Example 1, wherein the at least one electrode array comprises a flat thin conductive film wrapped around at least a portion of the elongate tubular body.

Example 6 relates to the depth electrode according to Example 5, wherein the thin conductive film is wrapped around an entire circumference of the elongate tubular body.

Example 7 relates to the depth electrode according to Example 1, wherein the at least one lumen comprises at least two lumens, wherein each of the at least two lumens are substantially parallel to each other.

Example 8 relates to the depth electrode according to Example 1, further comprising an optical fiber disposed within the at least one lumen.

Example 9 relates to the depth electrode according to Example 1, wherein the at least one lumen is constructed and arranged to allow for passage therethrough of a fluid, particulates, a procedural device, tissue, a treatment composition, or a medication.

Example 10 relates to the depth electrode according to Example 1, further comprising at least one electrical component coupled to the at least one electrode array, wherein the at least one electrical component is disposed between the tubular body and the at least one electrode array.

Example 11 relates to the depth electrode according to Example 1, further comprising at least one electrical component coupled to the at least one electrode array, wherein the at least one electrical component is disposed at a location external to the tubular body.

In Example 12, a depth electrode comprises an elongate tubular body, at least one lumen defined through a length of the elongate tubular body, a rotatable elongate structure disposed within the at least one lumen, an opening defined in a wall of the elongate tubular body, wherein the opening is in fluidic communication with the at least one lumen, and a deployable flat electrode array operably coupled to the rotatable elongate structure, wherein the deployable flat electrode moves between an undeployed configuration and a deployed configuration through the opening via rotation of the rotatable elongate structure.

In Example 13, a depth electrode comprises an elongate, lumenless tubular body, a distal cap disposed at a distal end of the elongate tubular body, and at least one electrode array disposed on an outer surface of the elongate tubular body and an outer surface of the distal cap, such that the at least one electrode array extends from the outer surface of the elongate tubular body to the outer surface of the distal cap.

In Example 14, a depth electrode comprises an elongate tubular body, and at least one electrode array disposed at least partially within the elongate tubular body, the at least one electrode array comprising a plurality of electrode contacts extending axially out of the elongate tubular body.

Example 15 relates to the depth electrode according to Example 14, further comprising at least one lumen defined through a length of the elongate tubular body.

In Example 16, a cortical electrode comprises an elongate structure, a thin film electrode array coupled to a distal end of the elongate structure, and a coupling structure coupled to a proximal end of the elongate structure, wherein the coupling structure is coupleable to an external connector.

Example 17 relates to the cortical electrode according to Example 16, wherein the elongate structure comprises at least one microwire.

Example 18 relates to the cortical electrode according to Example 16, wherein the elongate structure comprises a conductive thin film elongate structure.

Example 19 relates to the cortical electrode according to Example 16, wherein the thin film electrode array comprises at least two electrode contacts.

Example 20 relates to the cortical electrode according to Example 16, wherein the thin film electrode array comprises a rounded edge.

Example 21 relates to the cortical electrode according to Example 16, wherein the coupling structure comprises a conductive thin film coupling structure.

In Example 22, a shielding sheath comprises an elongate sheath body comprising a conductive material, a first opening at a first end of the elongate sheath body, a second opening at a second end of the elongate sheath body, a lumen disposed through a length of the elongate sheath body, wherein the lumen is in fluid communication with the first and second openings, a first attachment structure associated with the first opening, and a second attachment structure associated with the second opening.

Example 23 relates to the shielding sheath according to Example 22, wherein the conductive material is constructed and arranged to shield external radiofrequency.

In Example 24, a positionable cortical electrode comprises a thin film pad, a plurality of electrode contacts disposed in the thin film pad, and a plurality of flexibility openings defined in the thin film pad, wherein each of the plurality of flexibility openings are constructed and arranged to impart flexibility on the thin film pad.

Example 25 relates to the positionable cortical electrode according to Example 24, wherein the thin film pad comprises a first thin film layer and a second thin film layer, wherein the plurality of electrode contacts are disposed between the first and second thin film layers.

Example 26 relates to the positionable cortical electrode according to Example 25, further comprising a plurality of contact openings defined in the first thin film layer, wherein one of the plurality of electrode contacts is accessible via one of the plurality of contact openings.

Example 27 relates to the positionable cortical electrode according to Example 25, wherein the each of the plurality of flexibility openings are defined in the first and second thin film layers.

Example 28 relates to the positionable cortical electrode according to Example 24, wherein the plurality of flexibility openings comprise directional flexibility openings.

Example 29 relates to the positionable cortical electrode according to Example 24, wherein the thin film pad comprises a rounded edge.

In Example 30, a deployable elongate array device comprises at least two deployable elongate electrode bodies, a plurality of electrode contacts disposed on each of the at least two deployable elongate electrode bodies, and a rotatable joint rotatably coupled to each of the at least two deployable elongate electrode bodies, wherein the at least two deployable elongate electrode bodies are positionable in an aligned configuration and a deployed configuration.

Example 31 relates to the deployable elongate array device according to Example 30, wherein the at least two deployable elongate bodies are positionable in the aligned and deployed configurations via rotation around the rotatable joint.

In Example 32, a method of positioning an electrode tail under a patient's scalp comprises implanting an electrode through an implantation incision in the patient's scalp and a burr hole in a skull, inserting a distal end of a tunneling catheter through the implantation incision, urging the distal end toward a desired exit location in the patient's scalp, making an exit incision at the desired exit location, urging the distal end of the tunneling catheter out of the exit incision, urging a guidewire distally through a lumen of the tunneling catheter such that the guidewire extends from the implantation incision through the exit incision, removing the tunneling catheter while retaining the guidewire in position, attaching the electrode tail of the electrode to a proximal end of the guidewire, urging the guidewire distally through the exit incision until a distal end of the electrode tail is position through the exit incision, removing the electrode tail from the guidewire, and attaching skin at the exit point to the electrode tail.

In Example 33, a method of implanting an intracranial electrode array comprises forming first and second holes in a skull of a patient, inserting a guidewire through the first hole, urging the guidewire distally toward and through the second hole such that the guidewire is disposed through the first and second holes, urging an introduction sheath distally over the guidewire to a target intracranial position, positioning the intracranial electrode array at the target intracranial position via the introduction sheath, and removing the introduction sheath and the guidewire.

Example 34 relates to the method according to Example 33, further comprising adjusting a final position of the intracranial electrode array.

Example 35 relates to the method according to Example 34, wherein the adjusting the final position of the intracranial array comprises using a tool disposed through the first or second hole.

Example 36 relates to the method according to Example 33, wherein the positioning the intracranial electrode array at the target intracranial position via the introduction sheath further comprises positioning the intracranial electrode array in the introduction sheath prior to urging the introduction sheath distally to the target intracranial position.

Example 37 relates to the method according to Example 33, wherein the positioning the intracranial electrode array at the target intracranial position via the introduction sheath further comprises urging the intracranial electrode array into and through the introduction sheath after urging the introduction sheath distally to the target intracranial position.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a perspective view of a cortical electrode with magnets, according to one embodiment.

FIG. 2 is a perspective view of another cortical electrode with magnets, according to a further embodiment.

FIG. 3A is a perspective view of a cortical electrode with a laminate piezo film, according to one embodiment.

FIG. 3B is a cross-sectional side view of the cortical electrode of FIG. 3A, according to one embodiment.

FIG. 3C is a representational depiction of vibration waves created during use of the cortical electrode of FIG. 3A, according to one embodiment.

FIG. 4A is a perspective view of a cortical electrode with visualization markers, according to one embodiment.

FIG. 4B is a side view of a standard voltage reader for use with the cortical electrode of FIG. 4A, according to one embodiment.

FIG. 5 is a perspective view of a depth electrode with a spiral-shaped electrode array, according to one embodiment.

FIG. 6 is a perspective view of a depth electrode with four elongate electrode arrays, according to one embodiment.

FIG. 7 is a perspective view of a lumen-less depth electrode with four elongate electrode arrays, according to one embodiment.

FIG. 8A is a perspective view of a depth electrode with a contact array sheet prior to positioning the sheet on the electrode, according to one embodiment.

FIG. 8B is a perspective view of the depth electrode of FIG. 8A with the contact array sheet wrapped around the electrode body, according to one embodiment.

FIG. 9A is a perspective view of a depth electrode with a stacked electrode array, according to one embodiment.

FIG. 9B is a side view of the stacked electrode array of FIG. 9A, according to one embodiment.

FIG. 10 is a perspective view of a lumen-less depth electrode with four elongate electrode arrays having contacts coupled to microwires, according to one embodiment.

FIG. 11A is a perspective view of a depth electrode with four electrode arrays disposed within the tubular body with contacts extending therefrom, according to one embodiment.

FIG. 11B is a expanded, cross-sectional view of one trace coupled to one contact of the depth electrode of FIG. 11A, according to one embodiment.

FIG. 12 is a perspective view of a depth electrode with a body comprised of four contact arrays, according to one embodiment.

FIG. 13 is a perspective view of a depth electrode with a body comprised of four contact arrays and a lumen defined therethrough, according to one embodiment.

FIG. 14 is a side view of a flat depth electrode, according to one embodiment.

FIG. 15 is a top view of a cortical electrode, according to one embodiment.

FIG. 16 is a top view of another cortical electrode, according to another embodiment.

FIG. 17 is a cross-sectional side view of the internal components of an external connector, according to one embodiment.

FIG. 18A is a perspective view of one casing portion of the external connector of FIG. 17, according to one embodiment.

FIG. 18B is a perspective view of the second casing portion of the external connector of FIG. 17, according to one embodiment.

FIG. 18C is a side view of external wires that are coupleable to the external connector of FIG. 17, according to one embodiment.

FIG. 19 is a perspective view of a shield sheath, according to one embodiment.

FIG. 20 is a perspective view of a shielding head cover, according to one embodiment.

FIG. 21A is a schematic depiction of a tunneling catheter to position an electrode tail out of an exit point in a patient's scalp, according to one embodiment.

FIG. 21B is a distal end of a magnetic navigation tool, according to one embodiment.

FIG. 21C is a full perspective view of the magnetic navigation tool of FIG. 21B, according to one embodiment.

FIG. 22 is a perspective view of a cooling mat, according to one embodiment.

FIG. 23 is a perspective view of a cortical electrode having cooling fluid channels defined therein, according to one embodiment.

FIG. 24 is a perspective view of a closure device, according to one embodiment.

FIG. 25A is a top view of a cortical electrode array, according to one embodiment.

FIG. 25B is an expanded top view of the cortical electrode array of FIG. 25A, according to one embodiment.

FIG. 26 is a top view of another cortical electrode array, according to a further embodiment.

FIG. 27 is a perspective view of a depth electrode with an optic fiber disposed through the lumen of the electrode, according to one embodiment.

FIG. 28 is a perspective view of an electrode array pad for placement on a patient's foot, according to one embodiment.

FIG. 29 is a front view of an electrode array band, according to one embodiment.

FIG. 30 is a perspective of a depth electrode having a lumen that can be used for the passage of various materials and/or devices, according to one embodiment.

FIG. 31A is a schematic depiction of an x-ray image of a person's hands.

FIG. 31B is a perspective view of an electrode array pad for placement on a patient's hand, according to one embodiment.

FIG. 32 is a schematic depiction of an inflatable body for use with a cortical electrode array, according to one embodiment.

FIG. 33A is a perspective view of an electrode array pad, according to one embodiment.

FIG. 33B is an expanded side view of the electrode array pad of FIG. 33A, according to one embodiment.

FIG. 34A is a schematic view of a deployable electrode array device deployed in a patient's brain, according to one embodiment.

FIG. 34B is a top view of the deployable electrode array device of FIG. 34A in its deployed configuration, according to one embodiment.

FIG. 34C is a top view of the deployable electrode array device of FIG. 34A in its undeployed or retracted configuration, according to one embodiment.

FIG. 34D is a top view of another deployable electrode array device in its deployed configuration, according to another embodiment.

FIG. 35 is a side view of a die press, according to one embodiment.

FIG. 36 is a cross-section side view of a contact of an electrode array, according to one embodiment.

FIG. 37 is a cross-section side view of a contact of another electrode array, according to another embodiment.

FIG. 38 is a schematic view of a method of deploying an electrode array in a patient's brain, according to one embodiment.

FIG. 39 is a top view of an electrode array, according to one embodiment.

FIG. 40A is a schematic view of an electrode tail extending out of an exit incision in the back of a patient's head.

FIG. 40B is a perspective view of an electrode tail positioning and attachment device, according to one embodiment.

FIG. 40C is a perspective view of another electrode tail positioning and attachment device, according to another embodiment.

FIG. 41 is a perspective of a depth electrode having a deployable electrode array that is extendable out of an opening in the electrode body, according to one embodiment.

FIG. 42 is a side cross-sectional view of the internal components of an thin-film electrode array, according to one embodiment.

FIG. 43A is a side view of a depth electrode hub having a sensor disposed therein, according to one embodiment.

FIG. 43B is a side view of the sensor that is positionable within the depth electrode hub of FIG. 43A, according to one embodiment.

FIG. 44 is a cross-sectional axial view of a coated wire, according to one embodiment.

### DETAILED DESCRIPTION

The various embodiments disclosed or contemplated herein relate to improved systems, devices, and methods, and various components thereof, for recording neurological signals in the human body. More specifically, the implementations relate to various systems and devices for monitoring, stimulating, and/or ablating brain tissue, and various components of such systems and devices. In certain exemplary implementations, the various systems and devices incorporate ultra-thin dielectric materials with conductive materials placed thereon, thereby resulting in multiple conductors in high density on the devices, which improves the resolution of signal gathering per channel.

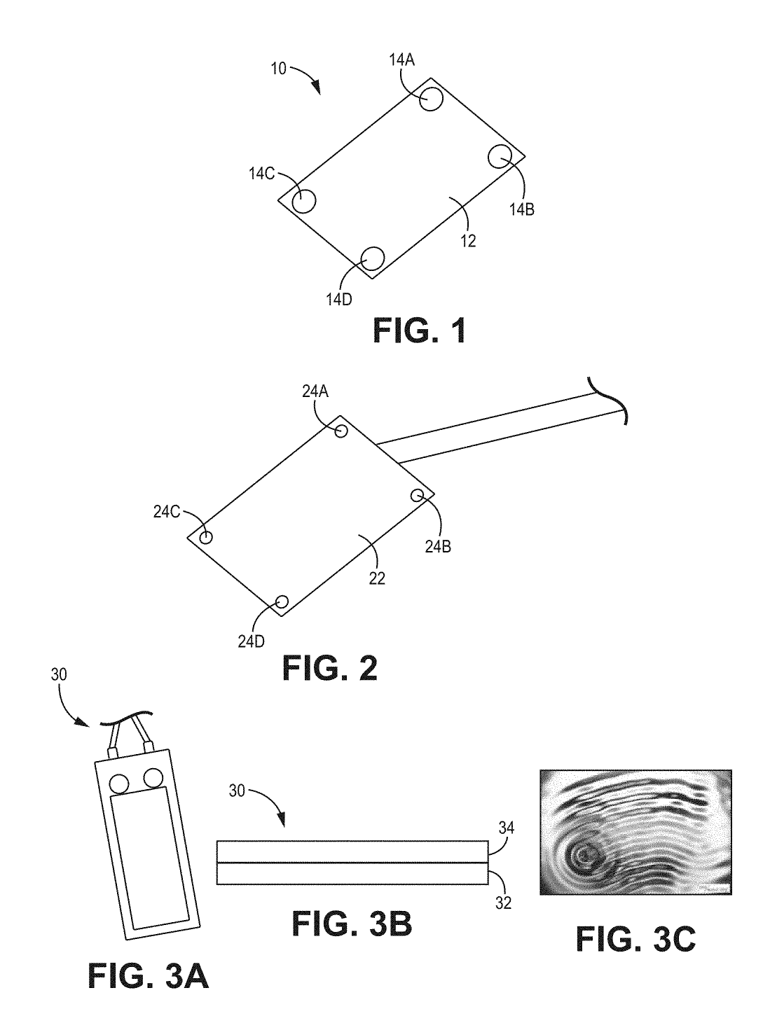

FIG. 1 depicts a positionable intracranial electrode array 10 that includes a pad 12 that has magnets 14A, 14B, 14C, 14D incorporated therein. In this specific embodiment, the array 10 has four magnets 14A, 14B, 14C, 14D positioned at or near the corners of the pad 12. Alternatively, the array 10 can have one, two, three, or five or more magnets positioned in any known fashion on the pad 12. According to one embodiment, each of the magnets 14A-D has a diameter of about 10 mm. Alternatively, each of the magnets 14A-D can range from about 5 mm to about 10 mm in diameter. Further, each of the magnets 14A-D can have a thickness ranging from about 1 to about 10 mm. Alternatively, the magnets 14A-D can have a thickness ranging from about 4 to about 6 mm.

In another example, the array 20 in FIG. 2 has a pad 22 with four 2 mm magnets 24A, 24B, 24C, 24D (in contrast to the 10 mm diameter magnets 14A-14D described above) positioned at or near the corners of the pad 22.

Further, the magnets 14A-D can be any known type of magnet for use in medical devices, including rare earth magnets, electromagnets, and other known magnets. Specific magnet examples that can be used in the embodiments herein include, for example, neodymium iron boron (“NdFeB”), samarium cobalt (“SmCo”), alnico, ceramic, and ferrite.

In one embodiment, the pad 12 is made of a polyimide material, such as Kapton® from DuPont®. Alternatively, the pad can be made of any other known flexible material for use in intracranial electrode arrays such that the pad can easily deform to match or otherwise accommodate the curvature of the cortical. In addition, the pad 12 in certain embodiments is a thin film pad. For purposes of this application, the term “thin film” can mean a microscopically thin layer material that is deposited onto a metal, ceramic, semiconductor or plastic base, or any device having such a component. Alternatively, for purposes of this application, it can also mean a component that is less than about 0.005 inches thick and contains a combination of conductive and dielectric layers. Finally, it is also understood, for purposes of this application, to have the definition that is understood by one of ordinary skill in the art. Further, it is understood that the pad 12 can be made according to any known process, including any known thin film processing.

In use, the electrode array 10, 20 can be implanted on a surface of the brain within the cranium of the patient. Once the array 10, 20 is implanted, this embodiment allows for positioning and re-positioning the array 10, 20 along the surface after implantation. That is, an external magnet (not shown) is disposed externally in relation to the cranium and, according to one implementation, positioned against the scalp of the patient such that the external magnet (not shown) magnetically couples with the magnets 14A-D, 24A-D on the pad 12, 22 disposed on the surface of the patient's brain. Once the external magnet (not shown) is magnetically coupled to the magnets 14A-D, 24A-D, the magnet (not shown) can be moved along the external surface of the cranium, thereby causing the pad 12, 22 to move along the surface of the brain within the cranium. This allows a surgeon or medical professional to position or re-position the array 10, 20 from outside the cranial structure, in some embodiments by using matched magnetic fields.

It is understood that the external magnet (not shown), in certain embodiments, is a neodymium magnet. Alternatively, the external magnet can be any known magnet for use in medical devices and related procedures.

Another positionable intracranial electrode array 30 embodiment is depicted in FIG. 3A. In this implementation, as best shown in FIG. 3B, the electrode array 30 has a laminate piezo film or layer 34 disposed on or integrated into the array base 32 as shown. In one embodiment, energy (such as, for example, a sine wave) can be passed through the film 34 such that the array 30 begins to vibrate. This vibration of the array 30 is shown visually as a set of waves in FIG. 3C. The vibration of the array 30 causes the surface friction between the array 30 and the surface of the brain to be reduced, thereby making it possible for the array 30 to more easily move in relation to the surface of the brain. Thus, a user or surgeon can actuate the piezo film 34 to use kinetics to alter the surface energy of the cranial fluid and thereby facilitate the movement of the array 30 in relation to the brain surface by causing the array 30 to become more “slippery” on the brain tissue surface. Again, this can allow a user to move or re-position the array 30 to different locations on the brain surface.

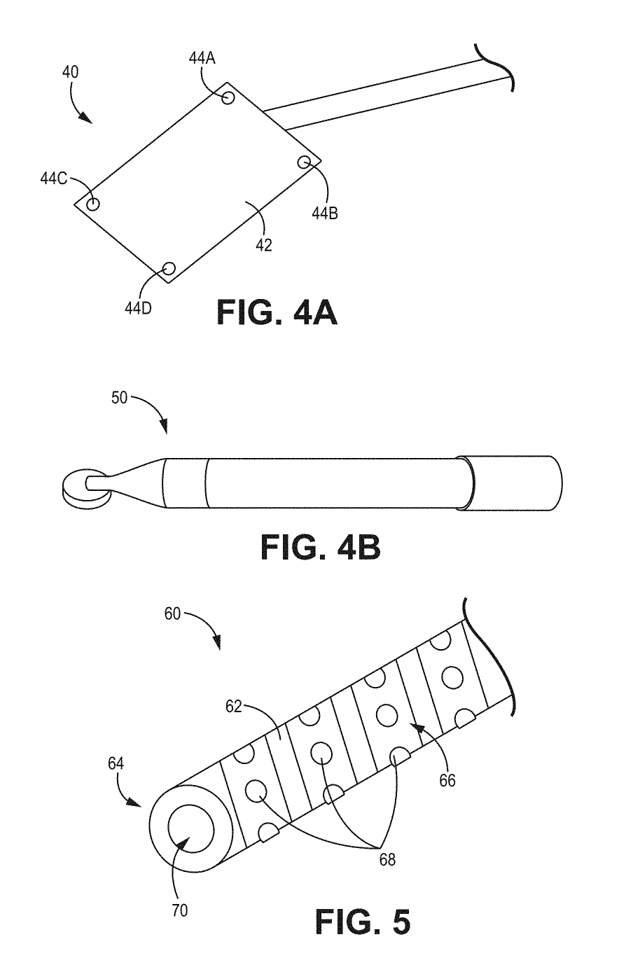

FIGS. 4A and 4B relate to a visualizable intracranial electrode array 40 as shown in FIG. 4A that includes a pad 42 having visualization markers 44A, 44B, 44C, 44D incorporated therein. In this specific embodiment, the four visualization markers 44A-D are four electrical coils 44A-D that are positioned at or near the corners of the pad 42. In use, the four coils 44A-D can be energized such that they can be detected by an external capacitive voltage reader positioned outside the cranium. That is, actuation of the coils 44A-D causes the coils 44A-D to generate a small electromagnetic field. Alternatively, the array 40 can have one, two, three, or five or more markers positioned in any known fashion on the pad 42. Alternatively, the markers 44A-D can be any known type of visualization marker for use in a medical device.

According to one embodiment, the visualization markers 44A-D can be detected using an external detecting device such as the known capacitive voltage reader 50 depicted in FIG. 4B. That is, the reader 50 has a tuned rf matched receiver coil that allows the reader 50 to detect the field generated by the coils 44A-D. In one embodiment as shown, the reader 50 is a RS Pro 457-8311 Magnetic Field Indicator/Detector, which is commercially available from Allied Electronics and Automation in Fort Worth, Tex. Alternatively, any known magnetic field reader or sensor can be used. As a result of the coils 44A-D and reader 50, the location of the array 40 is detectable without the use of imaging radiation or removal of the patient's head bandages.

In various embodiments, it is understood that any of the electrode array embodiments 10, 20, 30, 40 can incorporate any of the other features of those embodiments. That is, any of the electrode arrays 10, 20, 30, 40 depicted in FIGS. 1-4A can incorporate any one or more of the features or components that are disclosed in relation to the other array embodiments 10, 20, 30, 40. As such, the array 10 can also have a laminate piezo film 34 similar to the film in the array 30 and/or one or more visualization markers similar to the visualization markers 44A-D in the array 40.

In other implementations, the electrodes incorporated into the various systems herein can be depth electrodes (instead of thin electrode pads such as the pads 12, 22 discussed above) having thin films having conductive films (such as, for example, flexible circuits) incorporated therein. The various depth electrodes disclosed or contemplated herein (including the depth electrodes of FIGS. 5-14 as described in detail below) can not only detect the action potentials of active neurons in the brain, but can also detect the magnitude of the action potentials and the direction from which the action potentials are originating (the “vector” of the action potentials). Thus, in use, as few as three depth electrodes can be used to “triangulate” the location of the brain activity. As such, the use of the depth electrode embodiments as disclosed herein can reduce the number of intrusive electrodes or other devices required to be implanted into the brain in order to locate the target brain activity, thereby reducing trauma to the patient.

It is understood that any one of the depth electrode embodiments disclosed or contemplated below and elsewhere herein can incorporate any of the features of any of the other depth electrode embodiments herein in any combination.

FIG. 5 depicts a depth electrode 60 with a thin conductive film (also referred to herein as a “contact array”) 66 disposed thereon, according to one embodiment. The depth electrode 60 has tubular body (or “catheter”) 62 and a contact array 66 is disposed on the outer surface of the body 62. In this embodiment, the contact array 66 is a thin conductive film 66. The tubular body 62 of the electrode 60 in this implementation defines a lumen 70 that extends along the length of the body 62 from the distal end 64 to the proximal end (not shown).

In this exemplary electrode 60 (and any other depth electrode embodiment discussed below or otherwise contemplated herein), the lumen 70 can be used for various procedures or components. For example, the lumen 70 can be used for drug delivery, delivery of an imaging component (including, for example, a fiber optic or ultrasonic imaging component), delivery of a biopsy device, delivery of a temperature sensing device, delivery of an oxygen measuring device, cold (as described in further detail elsewhere herein) or warm therapy fluid delivery, delivery of a heating or cooling probe, or delivery of a laser treatment device. Alternatively, the lumen 70 can be used to provide fluidic access for delivery of any known medical component or treatment via the lumen 70. In certain alternative embodiments, the lumen 70 of the electrode 60 above (and the lumen of any other depth electrode embodiment herein) can be two or more lumens. It is understood that the two or more lumens can be used to provide fluidic access for delivery of any of the components, devices, or treatments discussed above in relation to the single lumen embodiments.

In this specific embodiment, the contact array 66 is a single thin conductive film 66 having a length that is wrapped around the body 62 in a spiral configuration as shown. As shown, the spiral configuration of the contact array 66 extends along the length of the body 62 to or almost to the distal end 64 of the body 62. That is, the distal end of the contact array 66 is positioned at or substantially adjacent to the distal end 64 of the tubular body 62 and extends proximally along the body 62 in the spiral configuration. In one embodiment, the spiral configuration provides flexibility to the electrode 60. The contact array 66 is a known circuit 66 having a plurality of contacts 68 disposed thereon as shown.

Each of the contacts 68 has an electrical connection or “trace” (not shown) operably coupled to the contact 68 that extends from the contact 68 to the proximal end of the contact array 66. Each trace (not shown) is disposed between the array 66 and the outer surface of the tubular body 62 such that the trace is not disposed within the tubular body 62 or the lumen 70 therein. According to one embodiment, the placement or positioning of the traces between the array 66 and the outer surface of the body 62 ensures that the lumen 70 can be used for any number of different procedures and other uses, because the traces are not positioned therein. It is further understood that each coupled contact 68 and trace (not shown) is electrically isolated from every other contact 68 and trace (not shown) pair. Further, it is understood that every depth electrode embodiment herein can have this feature relating to the electrical connections being outside of the tubular body.

It is understood that the depth electrode body 62 is an elongate tubular body 62 having a diameter ranging from about 0.5 mm to about 1.6 mm. Alternatively, the diameter can range from about 0.8 mm to about 1.4 mm. In a further embodiment, two specific diameter are about 0.84 mm and/or about 1.3 mm. In one implementation, the body 62 can be made of polyimide. Alternatively, the body 62 can be made of silicone, Pebax, Nylon, or any other polymeric material for use in a medical device. Further, the body 62 is a generally flexible body 62 such that the depth electrode 60 can be used in combination with a removable stylet or other similar device during use. That is, given the flexibility of the body 62, the stylet (not shown) can be inserted into the lumen 70 prior to positioning the electrode 60 such that the stylet provides additional support and/or stiffness to the body 62 such that the electrode 60 can be inserted into and positioned within the patient's brain as desired. Once the electrode 60 is positioned as desired, the stylet can be removed. It is further understood that any of these characteristics and features of the depth electrode 60 can apply to any of the depth electrode embodiments disclosed or contemplated throughout this specification. In addition, it is understood that, according to certain implementations, each of the depth electrode embodiments herein has an electrode body that is a single, unitary elongate structure, not multiple structures coupled together.

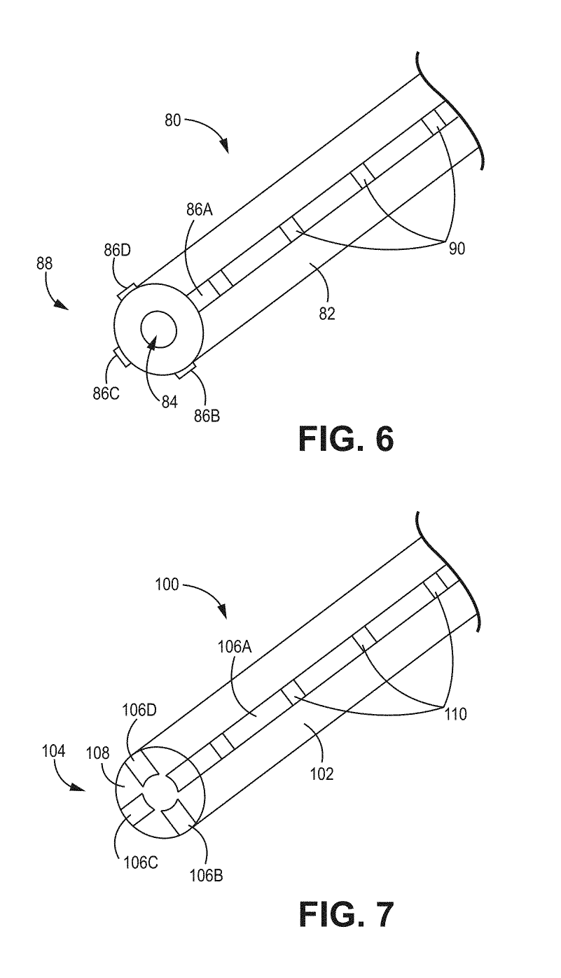

Another embodiment of a depth electrode 80 is depicted in FIG. 6. This electrode 80 has a tubular body 82, a lumen 84 defined therein that extends along the length of the body 82, and four contact arrays 86A, 86B, 86C, 86D disposed on the body 82. Each of the contact arrays 86A-86D in this specific example are longitudinal thin conductive films 86A-86D disposed on the outer surface of the body 82 and extending along the length thereof from at or substantially adjacent to the distal end 88 to some proximal portion (not shown) of the body 82. Each of the contact arrays 86A-86D is a known circuit having a plurality of contacts 90 disposed thereon as shown.

Yet another depth electrode 100 is depicted in FIG. 7, according to a further embodiment. In this specific implementation, the electrode 100 is substantially similar to the electrode 80 discussed above, except that it has no lumen. As such, the electrode 100 has a tubular body 102, a distal end 104, and four contact arrays 106A, 106B, 106C, 106D substantially similar to the contact arrays 86A-86D discussed above that are disposed on the body 102 as shown. Each of the contact arrays 106A-106D is a known thin conductive film having a plurality of contacts 110 disposed thereon as shown. In addition, the body 102 has a distal cap (or “cover”) 108 disposed at the distal end 104 of the body 102. Thus, the distal ends of the contact arrays 106A-106D extend distally beyond the outer circumference of the body 102 and are disposed on the distal cap 108 as shown.

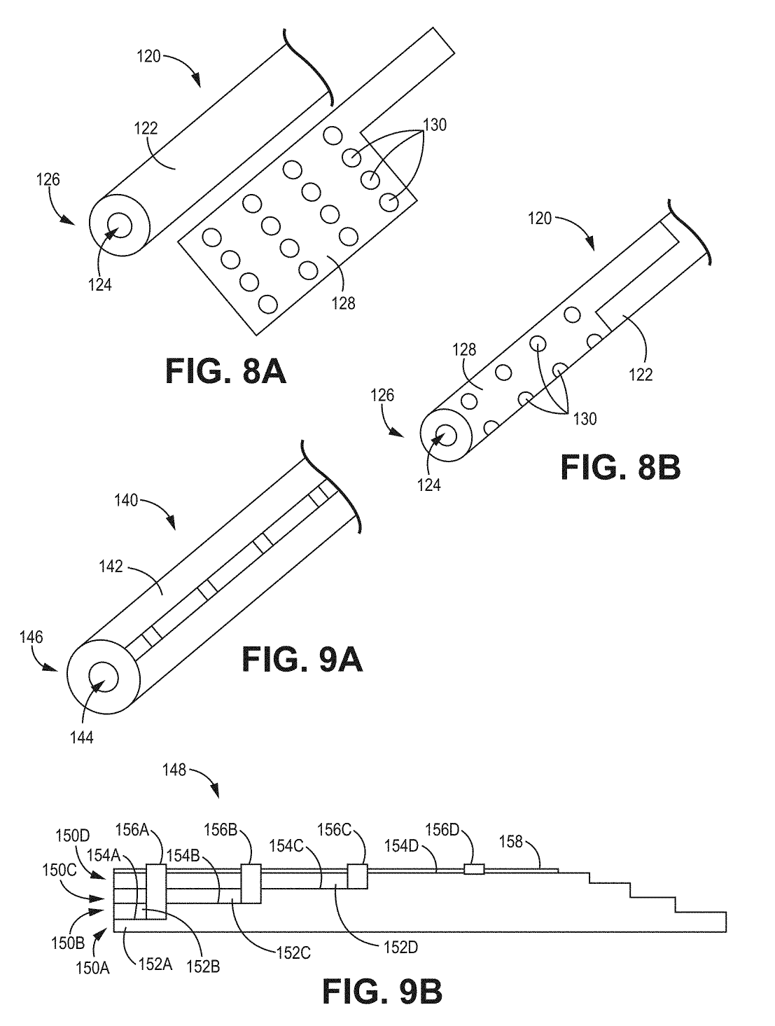

A further embodiment of a depth electrode 120 is depicted in FIGS. 8A and 8B. This electrode 120 has a tubular body 122, a lumen 124 defined therein that extends along the length of the body 122, and a distal end 126. In addition, the electrode 120 has a contact array film (or “sheet” or “wrap”) 128 that is depicted in FIG. 8A in its flat, unattached form. In the finished embodiment as shown in FIG. 8B, the contact array film 128 is wrapped or otherwise positioned around the outer surface of the body 122 such that the contacts 130 extend radially away from the body 122, thereby resulting in a set of contacts 130 disposed around substantially all of the 360 degrees of the circumference of the body 122. In one embodiment, the film 128 is single, unitary array 128 that is disposed on the body 122 such that it extends distally to or substantially adjacent to the distal end 126 of the body. Further, the film 128 extends proximally along the length of the body 122 to some proximal portion (not shown) of the body 122. It is understood that in certain embodiments, the contact array film 128 is a thin conductive film 128 such as a flexible circuit film.

Another embodiment of a depth electrode 140 is depicted in FIGS. 9A and 9B. This electrode 140, as best shown in FIG. 9A, has a tubular body 142, a lumen 144 defined therein that extends along the length of the body 142, a distal end 146, and a layered (or “stacked”) contact array (or “contact stack”) 148 disposed on the outer surface of the body 142. As best shown in FIG. 9B, the layered contact array stack 148 is made up of, in this particular example, four stacked longitudinal thin conductive films 150A, 150B, 150C, 150D layered on top of each other as shown, with the contacts 156A, 156B, 156C, 156D extending from the top dielectric layer 158 of the stack 148. That is, each of the contacts 156A-156D is positioned such that it extends through the top dielectric layer 158 such that each one extends from the top of the stack 148 as shown. It is understood that each thin film 150A-150D has a non-conductive portion 152A-152D and a conductive layer 154A-154D disposed on top of the non-conductive portion 152A-152D. Thus, each of the contacts 156A-156D is coupled to a conductive layer 154A-154D on one of the films 150A-150D as shown. More specifically, the contact 156A is electrically coupled to the conductive layer 154A on thin film 150A, the contact 156B is coupled to the conductive layer 154B on thin film 150B, the contact 156C is coupled to the conductive layer 154C on thin film 150C, and the contact 156D is coupled to the conductive layer 154D on thin film 150D. One advantage of this stacked configuration in which the conductive films 150A-150D are stacked vertically on top of one another is that the configuration can save space in comparison to a non-stacked circuit in which space is typically wasted underneath the contact array. As such, the stacked configuration of the electrode 140 results in higher surface packing density of the contacts 152A-152D in relation to the brain tissue. That is, the stacked contact array 148 allows for better use of scaffolding inner space and optimization of outer surface area for higher resolution of multiple contacts.

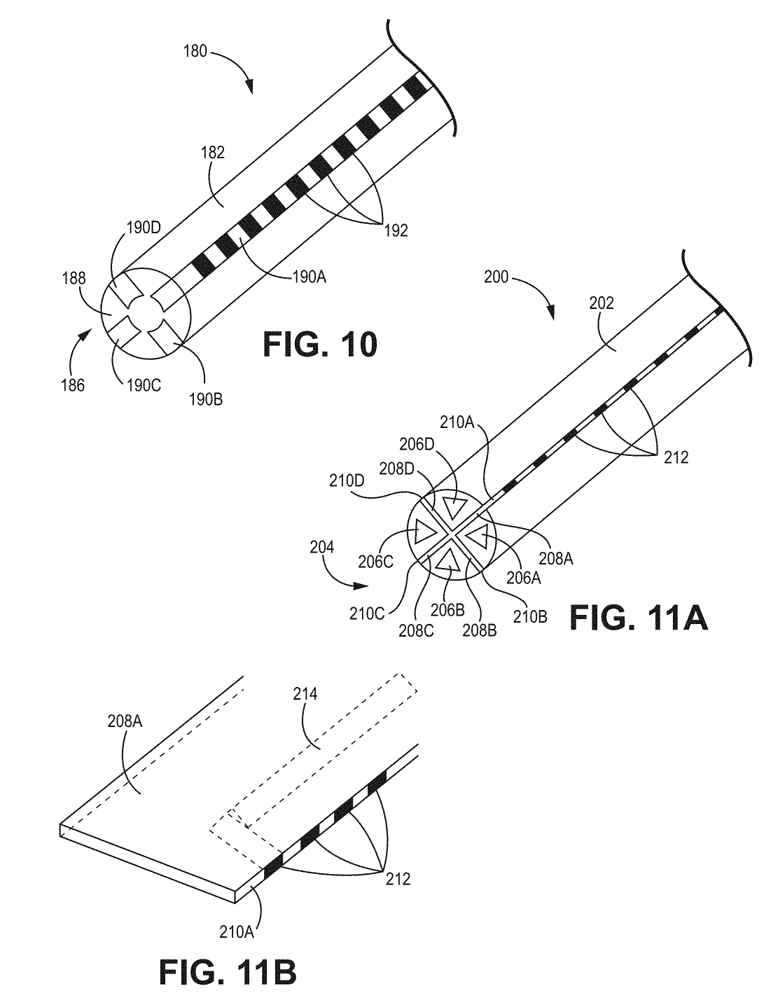

FIG. 10 depicts a depth electrode 180 in accordance with a further implementation. This electrode 180 has a tubular body 182, a distal end 186, a distal cap 188, and four contact arrays 190A, 190B, 190C, 190D disposed on the body 182 as shown. Each of the contact arrays 190A-190D, according to one exemplary embodiment, is a longitudinal thin conductive film 190A-190D disposed on the outer surface of the body 182 that extends along the length thereof from the distal end 186 to some proximal portion (not shown) of the body 182. In this specific implementation, each of the contact arrays 190A-190D has a plurality of contacts 192 disposed thereon as shown that are electrically coupled to small insulated wires (also referred to as “microwires”) (not shown), rather than exposed traces. The use of insulated wires (instead of traces) allows for the wires to be positioned close together or even in contact with each other without any risk of transfer of electrical charge across the wires, thereby making it possible to provide a greater number of contacts 192 along the length of each array 190A-190D. In one exemplary embodiment, the microwires have a diameter of about 0.001 inches (a gauge of about 49 or 50 AWG). Alternatively, the exemplary microwires can have a diameter ranging from about 0.0003 inches (a gauge of greater than 55 AWG) to about 0.002 inches (a gauge of about 44 AWG). Alternatively, the term “microwire” is intended to describe any wire that is considered to be a microwire by one of skill in the art.

In the embodiment as shown, the distal ends of the arrays 190A-190D extend distally beyond the outer circumference of the body 182 and are disposed on the distal cap 188 as shown. Alternatively, the body 182 can have a lumen disposed therethrough similar to other embodiments described above.

In an alternative embodiment for FIG. 10, the contact arrays 190A-190D are stacked arrays substantially similar to the stacked array 148 discussed above with respect to FIG. 9. In this embodiment, the stacked arrays 190A-190D allow for a greater number of contacts 192 due to the density provided by the stacking configuration.

In another embodiment, FIGS. 11A and 11B depict another depth electrode 200. As best shown in FIG. 11A, this electrode 200 has a tubular body 202, a distal end 204, four lumens 206A, 206B, 206C, 206D defined in the tubular body 202, each of which extends along the length of the body 202, and four longitudinal contact arrays 208A, 208B, 208C, 208D that are disposed within the body 202 with contacts 212 disposed on the outer edge 210A, 210B, 210C, 210D of each array 208A-208D such that the contacts 210 protrude from the outer surface of the body 202 as shown. As such, the contacts 210 are positioned on the cross-sectional area of each array 208A-208D. That is, the contact arrays 208A-208D in this implementation are embedded in or other disposed within the body 202 as shown, rather than positioned on the surface thereof. More specifically, as best shown in FIG. 11B, the contact array 208A is a thin conductive film 208A in which the contacts 212 are disposed on the edge 210A of the circuit 208A as shown. It is understood that while FIG. 11B depicts the contact array 208A, each of the other contact arrays 208B-208D has substantially the same configuration. Each individual contact 212 is electrically coupled to an individual trace 214 in the same or a similar fashion to the contact 212 electrical coupled to the trace 214 in FIG. 11B, with the trace 214 being embodiment in the thin conductive film 208A as shown. In certain implementations, the configuration of this electrode 200 can be varied by varying the thickness of the contact arrays 208A-208D.

In one implementation, each of the contact arrays 208A-208D is positioned within body 202 such that the array 208A-208D extends longitudinally along the length of the body 202 and further extends axially from a point substantially in the axial center of the body 202 to the outer surface of the body 202 as best shown in FIG. 11A. As such, each of the arrays 208A-208D is in contact with or substantially adjacent to the other arrays 208A-208D at or near the axial center of the body 202 as shown. Alternatively, instead of four arrays 208A-208D, there could be two arrays, each of which extends from the outer surface on one side of the body 202 through the axial center to the outer surface on the other side of the body 202. For example, in the embodiment depicted in FIG. 11A, 208A and 208C could constitute a single array and 208B and 208D could constitute a single array, with the two arrays intersecting at the axial center of the body 202. In a further alternative, each of the two arrays could be configured in a substantially 90 degree L-shaped configuration such that, for example, 208A and 208B could constitute two legs of a single array and 208C and 208D could constitute two legs of another single array.

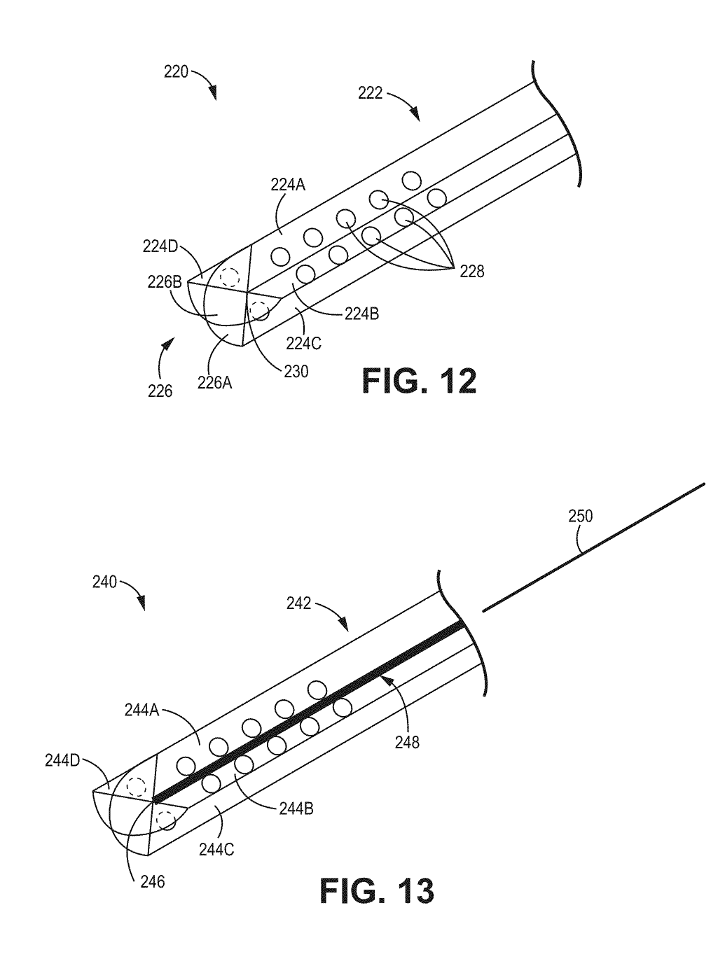

FIG. 12 depicts another embodiment of a depth electrode 220. In this embodiment, the body 222 is not a tubular shaped body, but instead is a body 222 that is made up of four longitudinal contact arrays 224A, 224B, 224C, 224D as shown. In this implementation, the body 222 has a tip 226 that is made up of two intersecting tip components 226A, 226B extending from the distal ends of the arrays 224A-224D. Each of the contact arrays 224A-224D has contacts 228 on both sides of the array as shown. Alternatively, each of the arrays 224A-224D can have contacts 228 on only one side. In one implementation, each array 224A-224D is a thin conductive film.

Each of the contact arrays 224A-224D extends longitudinally along the length of the body 222 and further extends axially from a point substantially in the axial center of the body 222 as shown. creating a set of four “paddles” in a “paddlewheel” configuration. As such, each of the arrays 224A-224D is in contact with or substantially adjacent to the other arrays 224A-224D at or near the axial center of the body 222 as shown. According to one embodiment, this central feature of the body 222 is the spine 230. Alternatively, instead of four arrays 224A-224D, there could be two arrays, each of which extends from the outer surface on one side of the body 222 through the axial center to the outer surface on the other side of the body 222. For example, 224A and 224C could constitute a single array and 224B and 224D could constitute a single array, with the two arrays intersecting at the axial center of the body 222 to create the spine 230. In a further alternative, each of the two arrays could be configured in a substantially 90 degree L-shaped configuration such that, for example, 224A and 224B could constitute two legs of a single array and 224C and 224D could constitute two legs of another single array, wherein each of the two single arrays 224A, 224B and 224C, 224D are joined at the spine 230.

In one implementation, the flexibility of the body 222 is determined by the thickness of the arrays 224A-224D. That is, the thicker the arrays 224A-224D, the stiffer or less flexible the body 222 of the electrode 220 is. In contrast, the thinner the arrays 224A-224D, the more flexible the body 222 is.

In use, this electrode 220 is delivered with a tubular structure (such as a sheath or catheter) around the electrode 220 to provide stiffness in the positioning and deployment thereof. Once the electrode 220 is positioned as desired, the tubular sheath is removed, leaving the electrode 220 positioned in the target area of the brain. With no tubular body, this electrode 220 has very low profile that is very non-intrusive to the brain tissue, which can allow for the electrode 220 to remain implanted for an extended time in the brain.

FIG. 13 depicts another embodiment of a depth electrode 240 having a body 242 that is made up of four longitudinal contact arrays 244A, 244B, 244C, 244D as shown in a configuration substantially similar to the body 222 discussed above. In accordance with one implementation, the body 242 and various components and features of the electrode 240 depicted in FIG. 13 is substantially similar to the body 222 and various components and features discussed above in relation to the electrode 220 of FIG. 12. Returning to FIG. 13, one difference in this implementation is that the spine 246 has a guidewire lumen 248 defined therethrough. As such, the lumen 248 is configured to receive a guidewire 250 such that the electrode 240 can be positioned over the guidewire 250 or the guidewire 250 can be inserted through the lumen 248. As such, this electrode 240 can be used in conjunction with a guidewire 250 to make the electrode 240 steerable.

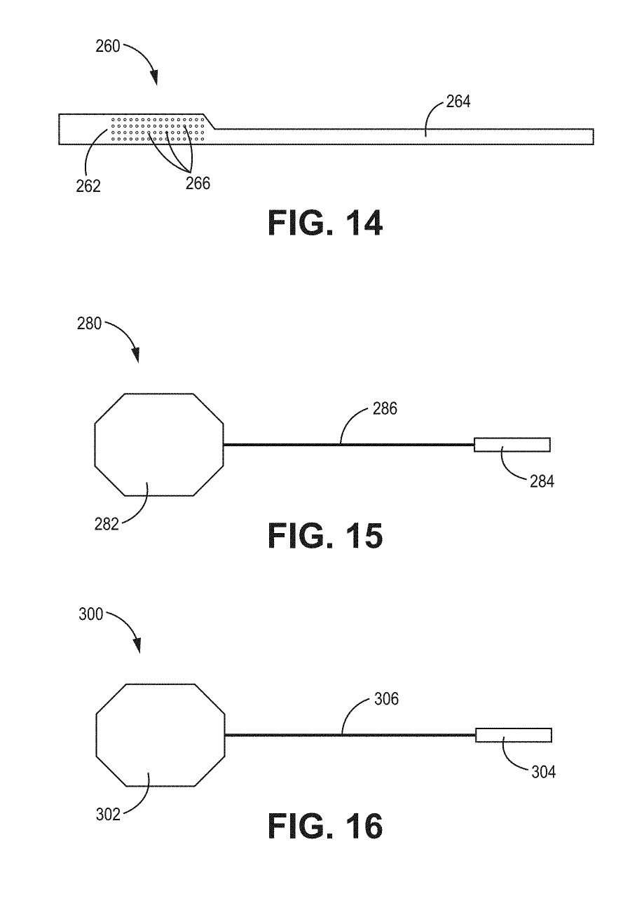

Other depth electrode implementations do not incorporate the supporting structure of a tubular body with a thin conductive film associated therewith like the above electrodes of FIGS. 5-13. Instead, one alternative is a depth electrode that utilizes the conductive film as the supporting structure (instead of a tubular body). For example, as shown in FIG. 14, a depth electrode 260 is provided that is a flat depth electrode 260. In this embodiment, the electrode 260 has a flat contact array 262 disposed at the distal end of an elongate flat component (also referred to herein as an “arm”) 264. In this implementation, both the arm 264 and the array 262 are formed from a thin conductive film. For example, in one specific embodiment, the arm 264 and array 262 are made of a polyimide film with electrode contacts 266 disposed on the array 262. Alternatively, the film can be any known thin conductive film. In addition, this electrode 260 can have a guidewire lumen (not shown) defined through the length of the electrode 260.

According to one implementation, the flat depth electrode 260 can be made using high resolution photolithography processing, which enables very fine trace of conductor material placed on the dielectric material.

One advantage of a flat or thin depth electrode such as the electrode 260 discussed above is that the minimal cross-section of such an electrode is less invasive in the cranial tissue, thereby reducing damage to the patient. Yet, despite the minimal cross-section, the use of the thin conductive film allows for maintaining a high packing density for the number of electrodes, thereby retaining the vector sensing capabilities discussed above. In contrast, as standard, non-flat depth electrodes enter the brain, cranial tissue is displaced (cut, moved, etc,), which can result in physiological counter-reactions in response to the intrusion, such as, for example, edema, bleeding, an immune system response, etc. Any such secondary response can degrade the quality of the signal.

In use, a guidewire (not shown) can be inserted through the guidewire lumen (not shown) prior to positioning the depth electrode 260 in the patient. The guidewire can provide structural rigidity for deployment and navigation into the tissue, allowing a user to urge the electrode 260 forward and navigate the brain tissue. It is understood that the guide wire can be a straight guide wire, a j-hooked guide wire, or a steerable guidewire that can have mechanical or electrical tip deflection. Alternatively, any known guidewire can be used. For removal of the electrode 260, the guidewire can be retracted and then the electrode 260 can simply be urged proximally out of the brain tissue.

In accordance with other implementations, new cortical electrodes for use with various systems as contemplated herein are also provided. One example of a cortical electrode 280 is depicted in FIG. 15, according to one embodiment. The cortical electrode 280 has a contact array 282 that is a conductive thin film 282 at a distal end of the electrode 280, and a proximal connector (also referred to as a “coupling component” or “tail”) 284 that is coupled to the contact array 282 via an elongate component (also referred to as a “body,” “line,” or “cable”) 286. In this embodiment, the elongate component 286 is one or more microwires 286. In one implementation, the contact array 282 has a plurality of contacts (not shown) disposed thereon in a fashion similar to the contact arrays discussed above with respect to the depth electrodes. The proximal connector 284 is configured to couple with an external connector such as the connector box discussed in additional detail below.

Another cortical electrode 300 is depicted in FIG. 16, according to a further embodiment. This electrode 300 has a contact array 302 that is a conductive thin film 302 at a distal end of the electrode 300, and a proximal connector 304 that is coupled to the contact array 302 via an elongate component 306. In this embodiment, the elongate component 306 is a conductive thin film 306. In one implementation, the contact array 302 has a plurality of contacts (not shown) disposed thereon in a fashion similar to the contact arrays discussed above with respect to the depth electrodes. The proximal connector 304 is configured to couple with an external connector such as the connector box discussed in additional detail below.

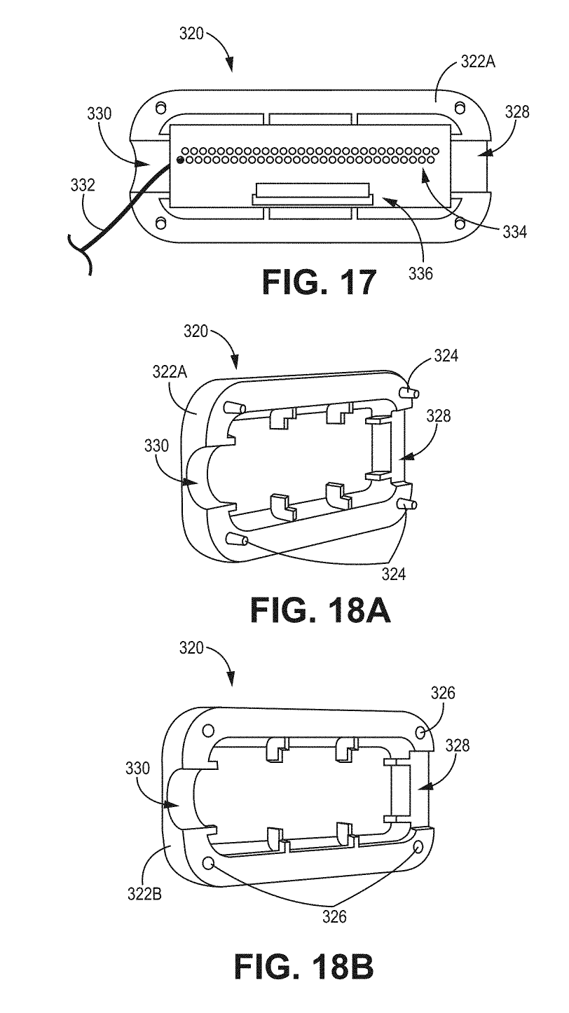

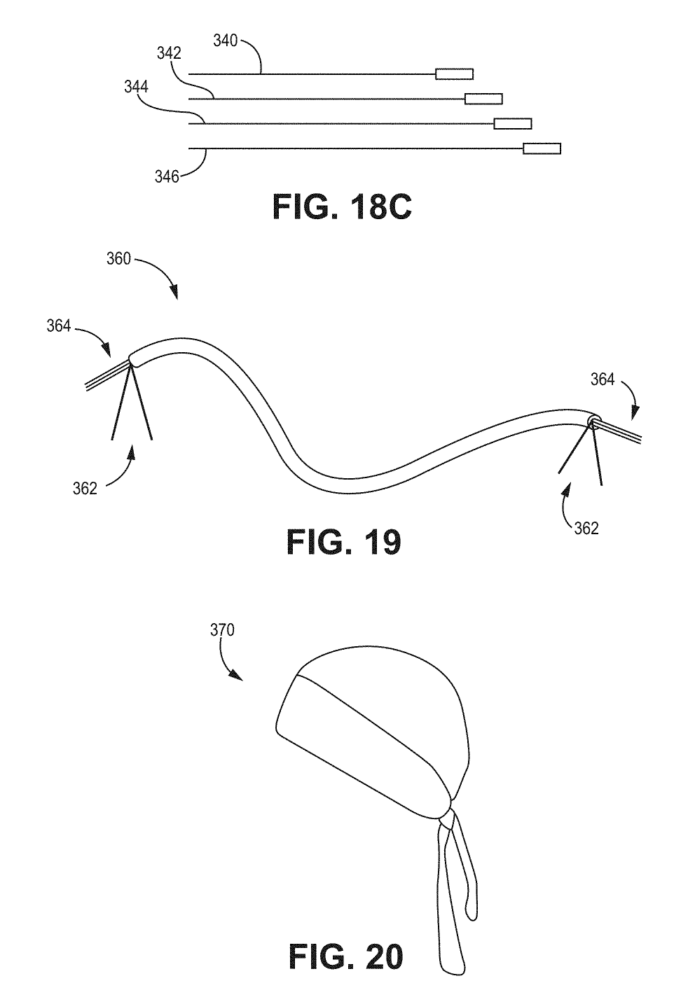

An external connector 320 is shown in FIGS. 17-18B, according to one embodiment. Note that FIG. 17 depicts the inner components of the connector 320, while FIGS. 18A and 18B depict the two portions of the outer casing 322A, 322B of the connector 320. As mentioned above, the connector 320 is coupled to the proximal connector of a cortical electrode (such as one of the cortical electrodes 280, 300 discussed above, for example) or any other known neural electrode. That is, the connector 320 provides the coupling of the neural electrode to the external components of the system, thereby conducting the signal from a flex structure to a wired structure.

In this implementation as best shown in FIG. 17, the connector 320 has a plurality of contacts 334 disposed within the casing 322 and a thin film connector (such as, for example, a zif connector) 336 that is electrically coupled to the contacts 334. In one embodiment, multiple external wires (such as wire 332, for example) can be coupled to the contacts 334 such that the wires are coupled to any thin film coupled to the thin film connector 336. As such, the connector 320 provides for coupling a conductive thin film to a set of external wires.

As best shown in FIGS. 18A and 18B, the casing 322 is made up of a two casing portions (or “halves”) 322A, 322B that couple together to form the external connector 320. The first casing portion 322A has four male projections 324 that are configured to mateably coupled with four openings or female components 326 on the second casing portion 322B such that the two portions 322A, 322B can mateably couple together. Further, each portion 322A, 322B has a portion of a conductive film opening 328 at one end and a wire opening 330 at the other end such that each of the openings 328, 330 is fully formed in each end of the connector 320 when the two portions 322A, 322B are coupled together. The conductive film opening 328 is configured to receive the conductive film or flexible circuit extending from the proximal end of the neural electrode according to any of the electrode embodiments disclosed or contemplated herein. The wire opening 330 is configured to receive the wires (such as wire 332 in FIG. 17, for example) that extend from the external controller of the various systems disclosed or contemplated herein.

In one embodiment as shown in FIG. 18C, a system and method is provided for assisting a user with coupling the correct external wires in the correct order to the external connector (such as the external connector 320 discussed above, for example). In this implementation, the various external wires are provided at different, predetermined lengths that indicate to the user the order in which they should be connected to the contacts of the connector (such as the connector 320 above). For example, in one embodiment, the shortest wire 340 is coupled to the connector first, followed by the next shortest wire 342, then the next 344, and then the next 346, etc. Alternatively, of course, the longest wire 346 can be coupled first, then the next longest 344, etc.

The various system embodiments disclosed or contemplated herein are sensitive to external radiofrequency (“RF”) noise that can interfere with the operation of the systems and/or devices. More specifically, the stray RF signals can attenuate the signals being recorded from the brain, thereby providing inaccurate information regarding those signals. FIGS. 19 and 20 depict two embodiments for shielding parts of the systems from such external RF noise. For example, FIG. 19 depicts a shield sock (or “sheath”) 360 that is made of a material having conductive material disposed therein. For example, in the specific embodiment as shown, the sheath 360 is a woven sheath made up of threads woven together, and at least one or more of those threads is made of a conductive material that is woven throughout the sheath 360. The conductive material acts as a shield to external RF noise. In one embodiment, the sheath 360 can be placed over any external wires or components 364 that extend from the external connector to the external components of the system, thereby shielding those wires or components from RF noise. In this specific embodiment, the sheath 360 also has attachment structures 362 disposed at each end of the sheath 360. More specifically, in this example, the attachment structures 362 are tie strings 362 to cinch or otherwise tighten the sheath 360 around the wires 364 and thereby attach the sheath 360 thereto. It is further understood that the shielding materials can be placed anywhere along the path of the various system embodiments or various components thereof that are disclosed or contemplated herein.

FIG. 20 depicts a shielding head cover 370 that can be worn by a patient to shield RF noise from affecting the electrodes and other components positioned inside the patient's head. The head cover 370 can be made of the same materials discussed above with respect to the sheath 360. In further implementations, the material could be formed into a wrap to wrap around the patient's head, sleeves that are used around the interface, a blanket placed over the recording structure, etc.

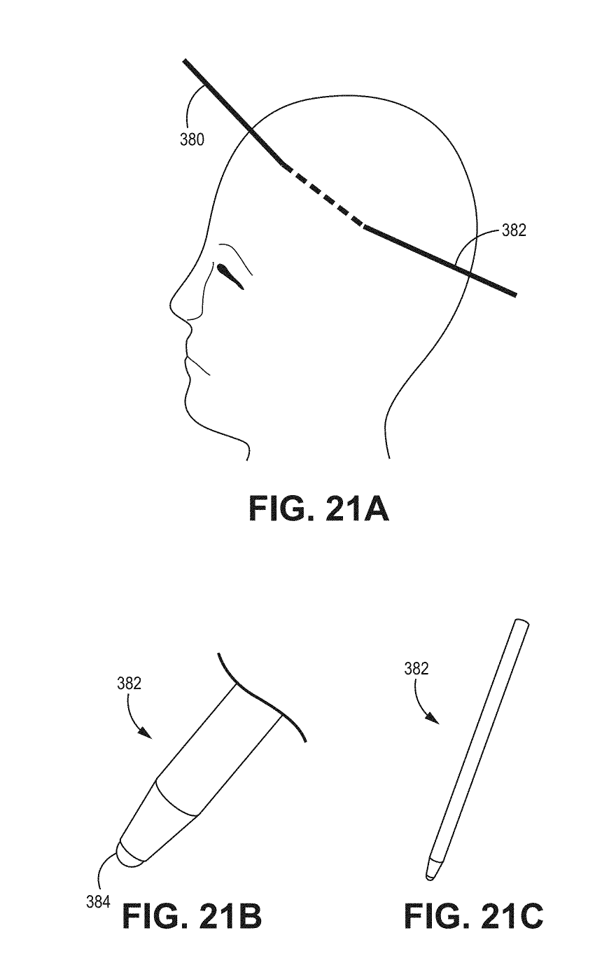

FIGS. 21A-21C disclose one embodiment of a tunneling catheter 380 for positioning the electrode tail between the burr hole in the patient's skull and the exit point out of the patient's scalp. It is understood that when a standard neural electrode is implanted in a patient for long-term use, the tail is positioned beneath the patient's scalp from the position of the burr hole on the skull to an exit point out of the patient's scalp at a location on the back of the patient's head (thereby reducing visibility and exposure of that tail as it exits from the patient's head). The known procedure for tunneling or positioning the tail under the patient's scalp involves the use of a rigid needle, which can be extremely painful for the patient. The tunneling catheter 380 as shown in FIG. 21A is a steerable catheter 380 that navigates between the patient's scalp and the skull, thereby helping to position the tail from the burr hole (not shown) to the exit point typically positioned on the back of the patient's head.

In use, according to one embodiment, the tunneling catheter 380 can be used in the following fashion to position the electrode tail out of an exit point in the patient's scalp that is typically positioned on the back portion of the patient's head. First, the cortical electrode (including, for example, any of the cortical electrode embodiments disclosed or contemplated herein) is positioned on the cortical tissue of the patient's brain as desired. Next, the tunneling catheter (such as catheter 380) is inserted under the scalp via the incision created for implantation of the electrode and the distal end is urged or steered toward the desired exit point for the electrode tail. Once the distal end of the tunneling catheter is urged under the scalp to the desired exit point, an incision is made at that exit point and the distal end of the catheter is urged therethrough. Once the tunneling catheter is positioned in this fashion, a guidewire or equivalent component is urged through the tunneling catheter from the proximal end to the distal end thereof. Once the guidewire is disposed through the tunneling catheter, the catheter is removed while maintaining the position of the guidewire such that the guidewire remains positioned under the scalp extending from the electrode insertion point to the tail exit point. At this point, the electrode tail is attached to the proximal end of the guidewire and the guidewire is urged distally out of the exit point (away from the electrode implantation point), thereby urging the electrode tail distally toward and ultimately out of the exit point. Alternatively, the tail can be urged or pushed through either the catheter or the tunnel created by the catheter using a steerable guidewire or the like.

In a further alternative, magnets can be used to provide navigation of the tail beneath the scalp. For example, the tail of the electrode catheter can have magnets (not shown) positioned thereon, and then an external magnetic navigation tool 382 with a magnet 384 positioned on the distal end as best shown in FIGS. 21B and 21C can be used outside the scalp to steer (push or pull) the catheter toward the exit point at the back of the head using magnetic forces.

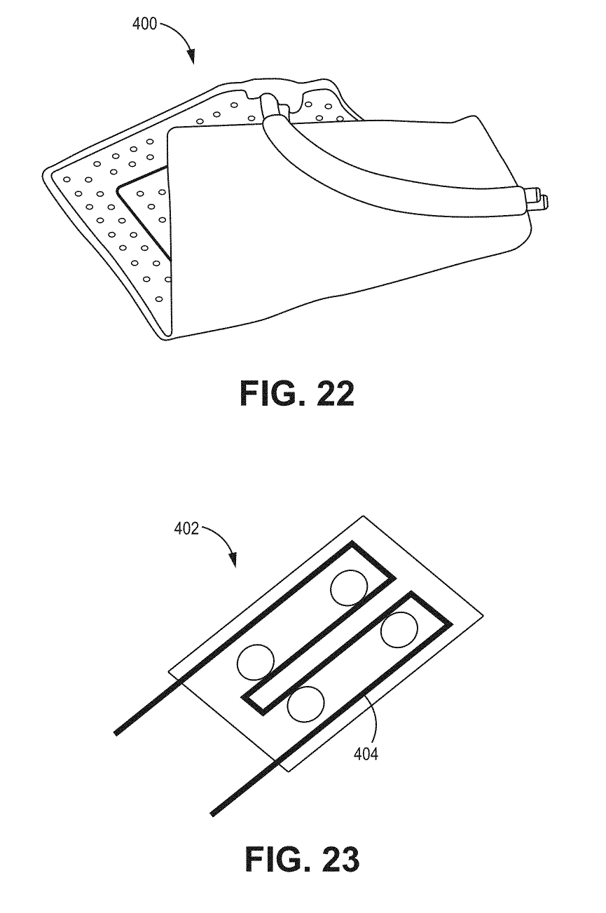

In other embodiments, a cooling feature can be provided with any of the systems disclosed or contemplated herein to help to cool the brain and thereby reduce the risk of seizures during a procedure or thereafter. That is, the brain tissue responds to temperature as a function of its ability to generate electrical pulses and communicate those pulses from neuron to neuron. As such, cooling the brain can reduce the risk of such communications resulting in seizures. A cooling component can be utilized to accomplish this. For example, FIG. 22 depicts a cooling mat 400 that can be placed on or over the patient's head. The cooling mat 400 can have chilled fluid disposed within or pumped through the mat 400, thereby reducing the temperature of the patient's head and brain.

Alternatively, as shown in FIG. 23, the cooling feature can be incorporated directly into the neural electrode 402. In this specific implementation, the electrode 402 has a fluid channel 404 defined within the electrode 402 through which cooling fluid can flow or be pumped, thereby helping to cool the patient's brain. In a further alternative, a cooling pad can be positioned adjacent to or coupled to or otherwise associated with the electrode (such as electrode 402), thereby providing the cooling action. In one exemplary embodiment, the cooling pad can be substantially similar to the pad 400 discussed above, except that the pad, for purposes of this specific embodiment, is sized to be positioned adjacent to, coupled to, or integral with any of the flat or pad electrode embodiments disclosed or contemplated herein. In one embodiment, the cooling pad can be made of a flexible thermoplastic material such as, for example, polyeythele. Alternatively, any known flexible polymeric material for use in such devices can be used. In those implementations in which a flexible polymeric material (including thermoplastic material) is used, the cooling channels can be formed by laminating two sheets of the material together with the channels formed therebetween. In certain embodiments, the cooling channel 404 is designed to allow the thermodynamic exchange of energy and thereby cool the tissue of the body (in this case, the cortical tissue). This cooling function can reduce edema and slow or stop an epileptic seizure that may occur during the procedure.

In a further embodiment, the chilled fluid can also be delivered via holes in the tubing to act as a sprinkler effect on the brain tissue. This turning on and off of seizure-related signals can help the doctor identify good/bad tissue and add another test for verification of correct location.

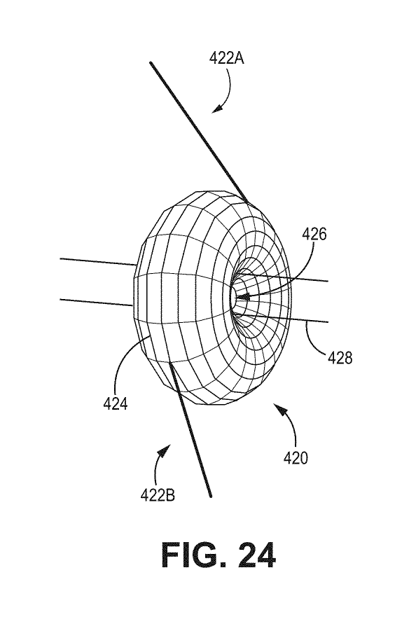

According to certain implementations as depicted in FIG. 24, a closure device 420 is provided for use in providing an support structure at the incision or exit point where the electrode tail 428 exits the scalp of the patient. This closure device 420 in this specific embodiment is a suturing corset 420 that operates in conjunction with two sutures 422A, 422B to reduce or narrow the exit opening through which the tail 428 is positioned. In one embodiment, the closure device 420 has a round body 424 with an opening 426 defined therein. In certain implementations, this configuration is “donut-shaped.” In use, a user can pull the two sutures 422A, 422B to reduce the size of the opening 426 in the body 424, thereby reducing the opening through which the tail 428 is positioned. In one embodiment, the body 424 can be biocompatible material used in other known port applications.

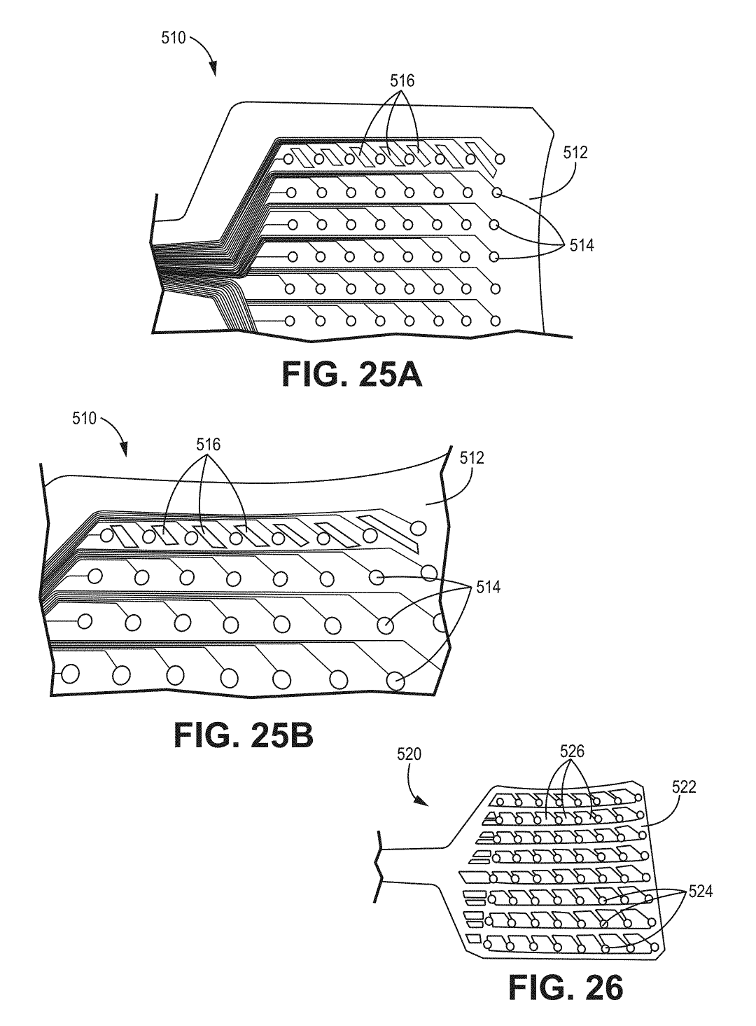

FIGS. 25A and 25B depict a positionable intracranial electrode array 510 that includes a thin-film pad (also referred to as an “electrode” or a “base”) 512 having electrodes (also referred to as “contacts”) 514 and openings 516 formed in the pad 512 adjacent to or around the electrodes 514. In this specific embodiment, the openings 516 in the array 510 are formed manually (by hand). Alternatively, FIG. 26 depicts a substantially similar electrode array 520 with a thin-film pad 522 having electrodes 524 and openings 526 formed by a laser in the pad 522. Alternatively, the openings 516, 526 can be formed with any known process or device, including waterjet, etching, etc.

It is understood that pads (such as pad 512) as discussed herein are sometimes referred to in the common vernacular in this area of technology as “electrodes” and electrodes (such as electrodes 514) as used herein are sometimes referred to as “contacts.” The terms and terminology used in this application are not intended to be limiting. The various components disclosed herein can be referred to by any known term in the art.

The pad 512 as shown in FIGS. 25A and 25B has only one row of openings formed in the pad 512 to depict the beginning of the opening formation (or material removal) process. It is understood that the remainder of the pad 512 can have openings formed therein in a fashion similar to that first row.

The formation of these openings 516, 526 removes excess material from the pads 512, 522, thereby imparting additional flexibility on the pads 512, 522 that was not present prior to formation of the openings 516, 526. The additional flexibility relative to standard pads (not shown) reduces the physical damage to tissue caused by the pads 512, 522 and reduces the overall physical footprint of the pads 512, 522 within the skull of the patient. Because of the flexibility, the body is less “aware” of the presence or is less likely to detect the presence of the pad (such as pad 512 or 522), thereby resulting in a lesser immune-response to the presence of the pad. Further, in accordance with another embodiment, the openings 516, 526 become attachment points for tissue growth across the pad 512, 522. That is, the openings 516, 526 can foster tissue growth across the pad 512, 522 after implantation. In addition, the openings 516, 526 can allow the tissue adjacent thereto to “breathe,” and thereby reduce the risk of negative reaction to the presence of the pad 512, 522 therein.

In accordance with one alternative, the openings 516, 526 can be formed selectively to create directional flexibility. That is, the openings 516, 526 can be purposely formed in a fashion that results in specific flexibility such that the pad 512, 522 is flexible in a specific, predetermined direction, thereby causing the pad 512, 522 to bend or deform to fit a specific curvature of the brain or other organ or body part.

In one embodiment, the thin-film pad (such as pad 512 or 522) is made of a polyimide material, such as Kapton® from DuPont®. Alternatively, the pad can be made of any other known flexible material for use in intracranial electrode arrays that can be modified by removing portions of the material as described herein. Further, it is understood that the pad (such as pad 512 or 522) can be made according to a known process of laminating two layers of polyimide (or any other known material for this purpose) together with the electrode contacts (also referred to as “traces) therebetween and then using a laser to expose the contacts. Alternatively, the pad (such as pad 512 or 522) can be made according to any known process.

In accordance with one implementation, the openings 516, 526 are formed in the pad 512, 522 after forming the pad 512, 522 according the process described above. Alternatively, the openings 516, 526 can be formed therein at any step in the process.

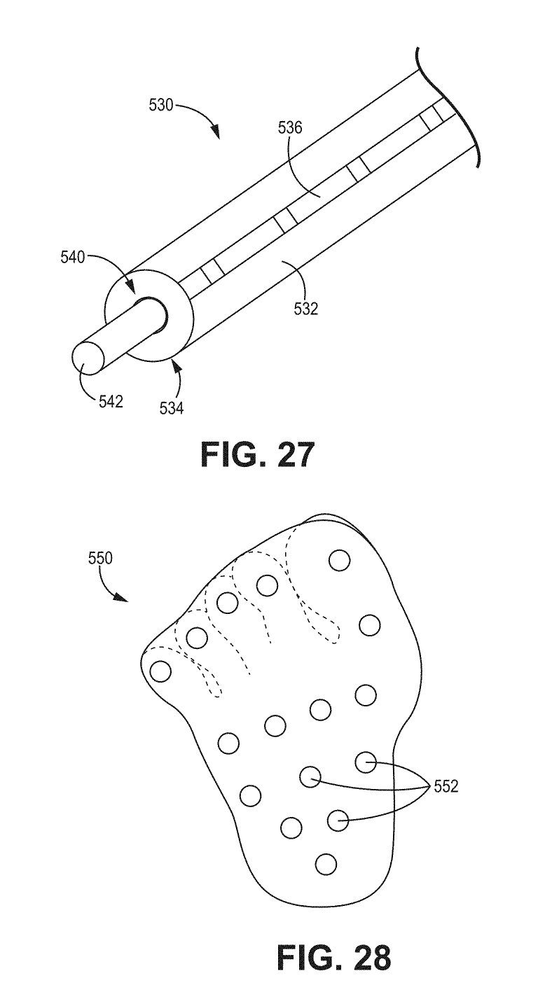

FIG. 27 depicts a depth electrode 530 with a contact array 536 disposed thereon, according to one embodiment in which the array 536 is a thin conductive film 536. The depth electrode 530 has tubular body (or “catheter”) 532 and the contact array 536 is disposed thereon. The tubular body 532 of the electrode 530 in this implementation defines a lumen 540 that extends along the length of the body 532 from the distal end 534 to the proximal end (not shown).

In this exemplary embodiment, the lumen 540 can be used for delivery of an optical fiber 542 through the lumen 540 for imaging purposes. That is, the optical fiber 542 can be used for viewing inside the brain tissue. More specifically, the optical fiber 542 can be used to navigate or place the electrode 530 by allowing the surgeon to see the areas surrounding the electrode 530 and steer the electrode 530 appropriately for navigating into and through the brain tissue or implanting the electrode 530 therein. Further, the optical fiber 542 can be used by a surgeon or other user for any other purpose for which a view of the area distal to the electrode 530 is visible, including, for example, examining the condition of the tissue in that area for various purposes, including monitoring the response of the tissue to the presence of the electrode 530.

One advantage of the use of an optical fiber (such as fiber 542) is the reduction or elimination of the need for exposing the patient to radiation (such as an MRI or X-ray) in order to capture a view of the interior of the brain or any portion of the brain tissue therein. The use of the optical fiber (such as fiber 542) to provide a view of the tissue through the lumen (such as lumen 540) can also be used to visually verify bleeding (rather than having to take the time to get an MRI for the same purpose).

Alternatively, certain of the thin film electrode array technologies can be used for other applications. For example, as shown in FIG. 28, in one embodiment, an electrode array pad 550 can be positioned on the underside of a patient's foot (or positioned so that the underside of the patient's foot is positioned thereon) such that certain characteristics of the foot can be monitored. In one specific example, the pad 550 can be used to monitor muscle contraction or EMG in diabetic patients, which can be used to quantify how the diabetic patient's foot is affected by the disease. Alternatively, the pad 550 and the contacts 552 can be used to detect the application of force or to detect the temperature of the foot. One advantage of the multiple sensor contacts (such as contacts 552) of a thin film electrode array (such as the pad 550) is that the multiple contacts can overcome the impedance of the dermal skin layer and thus make it possible to record and deliver electrical signals.

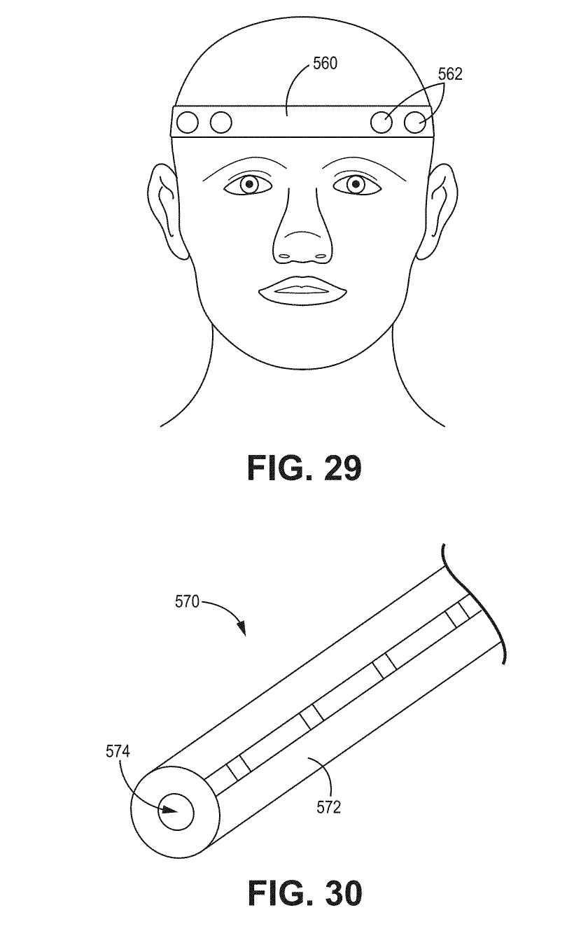

Yet another application for thin film electrode array technologies is depicted in FIG. 29, in which an electrode array 560 takes the form of an electrode array band 560 that can be positioned around the head of a patient as shown. In this embodiment, the band 560 has contacts 562 disposed on the band 560 at or near the forehead and temples of the patient. In one specific implementation, the electrode array band 560 can be used to monitor and ultimately diagnose concussions in athletes. For example, the band 560 can be placed on a patient or subject prior to participation in a contact sport (such as football, for example), including at the beginning of the season or prior to the subject ever having participated in the sport. This would establish a baseline reading of the subject's healthy brain. Subsequently, readings could be taken using the band 560 during a game, during the season, and/or at the end of the season to monitor any changes to the readings and determine if any such changes were caused by brain damage and/or concussions. In one embodiment, the band 560 could be used to determine whether any concussion has occurred and thereby use the band 560 to determine whether the subject is sufficiently healthy to continue to participate in the activity or needs to stop.

FIG. 30 depicts a further embodiment of a depth electrode 570 having a lumen 574 that extends along the length of the body 572 of the electrode 570. In this implementation, the lumen 574 is used as a passage for delivering fluids, particulates, or the like or taking samples of tissue or fluids for a biopsy or other purposes. For example, in certain implementations, the lumen 574 can be used for non-systemic treatment of the brain tissue through the delivery of an appropriate treatment fluid or particulate therethrough. In one specific exemplary embodiment, the brain tissue can be treated with a cooling treatment. More specifically, cool fluid is delivered to the brain tissue via the lumen 574 of the depth electrode 570 (or the lumen of any depth electrode disclosed or contemplated herein) to cool the brain tissue therewith. Alternatively, the lumen 574 can be used to take a sample for purposes of drug testing. In a further alternative, the lumen 574 can be used as a brain port for delivery of drugs for treatment of a tumor.

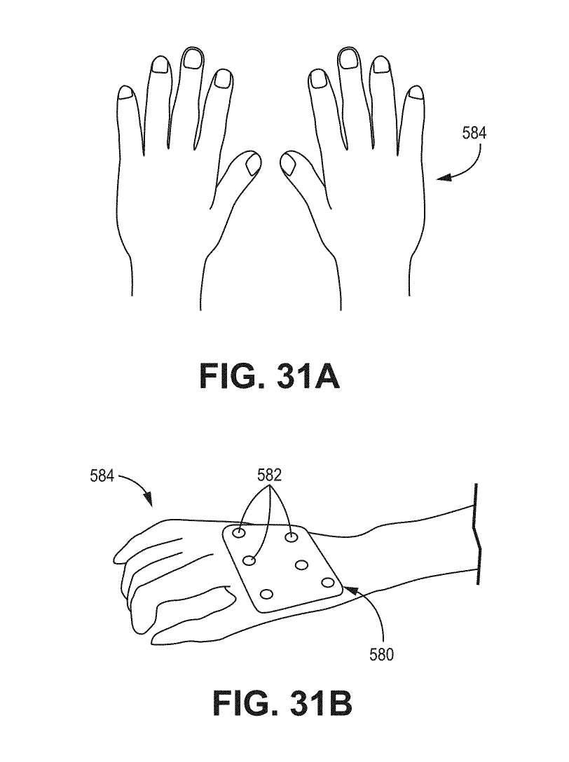

Additional alternative applications for thin film electrode array technologies are depicted in FIGS. 31A and 31B according to one embodiment in which an electrode array pad or band 580 can be positioned on a subject's hand 584 such that certain characteristics of the hand 584 can be monitored. The pad 580 has an array of electrodes 582 thereon as shown.

In one specific example, the pad/band 580 can be positioned on or over one or more joints or muscles of a subject's hand 584 and used to monitor electrical muscle activity in the arthritic subject. According to further implementations, the data relating to the electrical muscle activity can be compared to the subjective pain of the subject to translate the information into quantifiable data about how the electrical muscle activity relates to arthritic pain for the subject. It is understood that multiple points can be measured such that EMG mapping can be accomplished.

In a further specific example, the pad/band 580 can be positioned on the hand 584 to monitor through-the-skin EMG in Parkinson's patients to measure signals in the hand 584 relating to involuntary movements. According to one implementation, the measurement of these signals can be used to understand the timing of the involuntary movements. For example, a baseline of tremors can be established with combined data from the hand 584 and the brain by measuring the distance and timing of such signals in the brain and the hand 584.

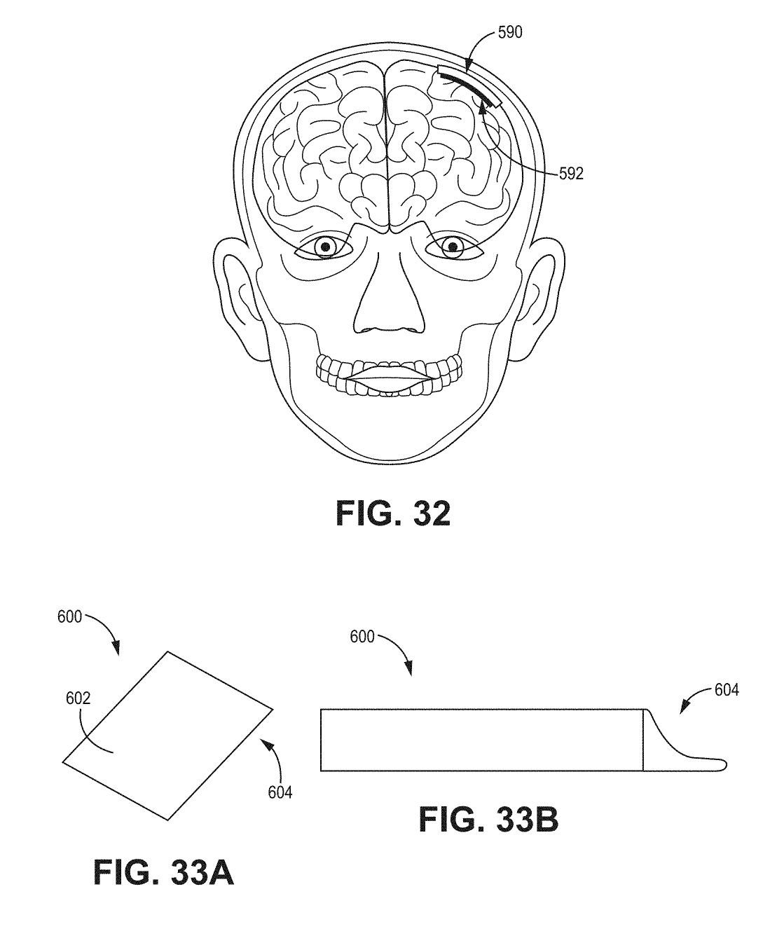

The various thin film electrode array embodiments disclosed or contemplated herein can be implanted on a surface of the brain within the cranium of the patient. Once the array is implanted, it is advantageous for the array to be in close contact with the surface of the brain. In one embodiment, as depicted in FIG. 32, upon or in conjunction with implantation of an electrode array (or “pad” or “electrode”) 592 according to any embodiment disclosed or contemplated herein, an inflatable balloon 590 can be positioned between the array 592 and the skull 594. The balloon 59 can be any known balloon of any known material for use in conjunction with treatment of the brain. According to one implementation, in use, the balloon 590 can be inflated to urge the array 592 against the surface of the brain, thereby increasing the contact of the array 592 with the brain surface and thereby lowering the impedance, which improves signal quality. Further, according to certain embodiments, the positioning of the balloon 590 to urge the electrode 592 against the surface of the brain tissue can allow for incomplete connections to be resolved by improving the contouring of the electrode 592 to the curvature of the cranial tissue via the force applied by the balloon 590. According to a further embodiment, the balloon 590 can also be used to deploy the array 592. That is, the balloon 590 can be implanted with the array 592 and can be inflated upon positioning in the desired location within the skull to cause the deployable array 592 to unroll or otherwise deploy on the surface of the brain. In certain embodiments, the balloon 590 can remain uninflated until needed. That is, the balloon 590 can remain uninflated until a seizure starts and can then be inflated to increase the contact of the array 592 with the brain surface, thereby improving the signal quality to ensure a clear reading and enhancing the ability to identify and map such a seizure.

Another embodiment of an electrode array 600 that includes a pad 602 is depicted in FIGS. 33A and 33B. In this implementation, the pad 602 has an edge 604 that has been modified to reduce the potentially sharp nature thereof. More specifically, the known electrode array pads in the art typically have a sharp edge: an edge that narrows essentially to a point and thereby creates a risk of the sharp edge of the pad cutting into the brain tissue or other tissue. In contrast, the pad 602 according to this implementation has a modified edge 604 that has been modified to reduce the sharp nature of the edge 604 and/or create a more rounded or “feathered” edge 604, thereby reducing or eliminating the risk of any tissue of the patient being cut or otherwise damaged. It should be noted that the process for creating the modified edge (such as, for example, the modified edge 604) of the electrode 602 can be an additive or subtractive process. That is, the edge modification process can be either the addition or removal of material to “soften” or otherwise modify the edge.

In one specific example, the modification process can relate to polishing or otherwise forming a radius or curvature onto the edge (such as edge 604). Another approach according to a further implementation would be to place an overlapping material around the edge (such as edge 604). According to another embodiment, the modifications to the modified edge 604 can be accomplished via laser ablation of the edge 604 to reduce or eliminate the sharp nature of the edge 604. Alternatively, any known method or process for edge modification can be used. In a further alternative, this modification can also be applied to the edges of each of the openings formed in the pad as described above with respect to FIGS. 25A-26.

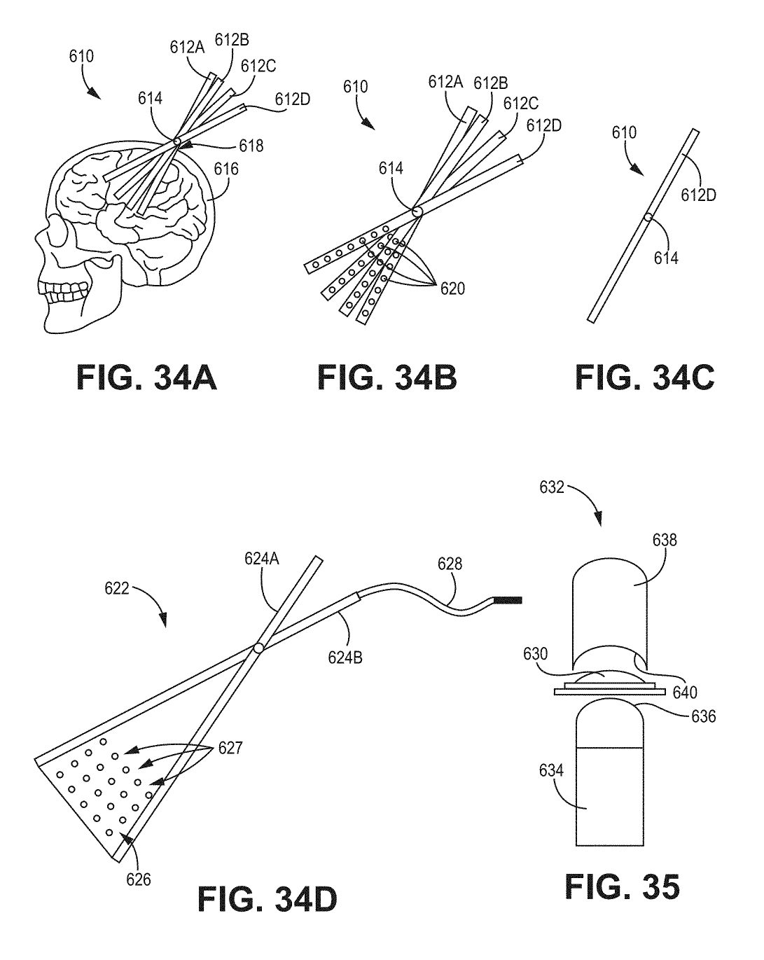

Further implementations of an electrode array 610 according to further embodiments are depicted in FIGS. 34A-34D. In one embodiment as shown in FIGS. 34A-34C, the array 610 is made up of two or more strip arrays 612A, 612B, 612C, 612D, wherein each array has multiple contacts 620. More specifically, in this specific embodiment, the array 610 is made up of four strip arrays 612A, 612B, 612C, 612D. Alternatively, the array 610 can have two strip arrays, three strip arrays, five strip arrays, or six or more strip arrays. The strip arrays 612A, 612B, 612C, 612D are rotatably coupled to each other at a relatively middle point along the length of each of the arrays 612A, 612B, 612C, 612D. That is, the four arrays 612A, 612B, 612C, 612D are rotatably attached to each other via a rotatable joint 614 at a generally midpoint of each array 612A, 612B, 612C, 612D such that the four arrays 612A, 612B, 612C, 612D can move between an aligned configuration (as best shown in FIG. 34C) in which the longitudinal axis of each of the four arrays 612A, 612B, 612C, 612D are substantially aligned with the others in such a fashion that the four axes are substantially parallel and a “fan” or deployed configuration as best shown in FIGS. 34A and 34B in which none of the four axes are parallel.

Alternatively, the rotatable joint 614 can be positioned at other points along the length of each strip array 612A, 612B, 612C, 612D other than the midpoint of each. In other words, in certain embodiments, the rotatable joint 614 can be positioned between the distal end and the midpoint of each of the strip arrays 612A, 612B, 612C, 612D. In a further alternative, the joint 614 can be positioned between the proximal end and the midpoint. According to various embodiments, the rotatable joint 614 can be positioned anywhere along the length of the strip arrays 612A, 612B, 612C, 612D.

According to one embodiment, the rotatable joint (such as joint 614) provides the unique feature of adjustable resolution in a minimally invasive electrode array. That is, the rotatable joint (such as joint 614) allows a user to adjust the resolution of the array 610 by moving the strips between their aligned and deployed configurations. That is, the two or more strip arrays (such as strips arrays like 612A, 612B) can be moved between their deployed configuration and their aligned configuration or any point in between such that the contacts (not shown) on the cranial tissue are more dispersed relative to each other (resulting in lower resolution) or closer to each other (resulting in higher resolution), respectively. In certain implementations, this adjustment feature can help to obtain precise definition or tissue margins between suspect tissue and healthy tissue. In accordance with certain exemplary embodiments, this array 610 can be adjusted after it has been positioned within the cranial tissue to achieve the desired resolution.

In use, the electrode array 610 can be implanted and deployed as follows, according to one implementation. First, the array 610 is placed in the aligned configuration (as best shown in FIG. 34C) such that the array 610 can be inserted through a surgical opening 618 in the skull 616 (as best shown in FIG. 34A) of the patient. The array 610 is then inserted through the opening 618. Once the array 610 is disposed through the opening 618 as desired, the array 610 can be moved into its deployed configuration as best shown in FIG. 34A. In the deployed configuration, the distal ends of the strip arrays 612A, 612B, 612C, 612D are disposed within the skull 616 in a fan-like spread that forms a grid of electrodes 620 in the brain tissue as shown. According to one embodiment, the rotatable joint 614 is lockable so that once the strip arrays 612A, 612B, 612C, 612D are disposed in the deployed configuration, they can be locked in that configuration until it is desired to remove the array 610 from the patient's skull 616. When it is time to remove the array 610, the rotatable joint 614 can be unlocked, the strip arrays 612A, 612B, 612C, 612D can be moved back into the aligned configuration and removed through the single opening 618.

In one embodiment, the joint 614 is any known rotatable coupling of two or more separate components such as strip arrays. Alternatively, instead of a rotatable joint 614, the strip arrays 612A, 612B, 612C, 612D can be independent, uncoupled arrays that are inserted through the opening 618 together, deployed into a deployed configuration, and then attached to each other via a clamp, an adhesive, or any other known mechanism to retain the strip arrays 612A, 612B, 612C, 612D in the deployed configuration.

A further alternative embodiment of a deployable array device 622 is depicted in FIG. 34D, in which the device 622 mimics an expandable handheld fan. This array device 622 has two elongate members (or “strips” or “rods”) 624A, 624B with a foldable film 246 extending between and attached to both of the members 624A, 624B such that the film 246 is expanded to its deployed configuration as shown in FIG. 34D when the two members 624A, 624B are moved into their deployed position in a similar fashion to that described above with respect to the device 610. In this embodiment, the electrode contacts 627 are disposed on the film (or “sheet” or “laminate”) 626 such that the contacts 627 are distributed across a fan-like spread within the patient's brain in a similar fashion that described above. In this embodiment, at least one of the elongate members 624A, 624B is coupled to a wire or elongate component 628 that can be coupled to an external controller or power source. The device 622 can be used in a similar fashion to the device 610 above.

In one implementation, any of the electrode arrays, including the thin-film electrode arrays, according to any of the embodiments disclosed or contemplated herein, can have “domed” or raised electrode contacts 630 as shown in FIG. 35. More specifically, each electrode 630 in any array embodiment can have a raised or domed configuration as shown. According to one embodiment, the domed contacts can be formed using a press 632 having a die 634 with a curved distal end 636 and a receiver 638 with a matching curved receptacle 640 into which the curved distal end 636 fits. As such, a contact 630 can be caused to be formed into a domed or raised configuration by positioning the contact 630 between the die 634 and receiver 638 as shown such that the die 634 can be pressed into the receiver 638 and thereby cause the contact 630 to take on a domed shape as shown.

It is understood that one implementation of the device to create the domed contacts could be a device having multiple dies 634 and receivers 638 disposed to match up with the contacts in an array.

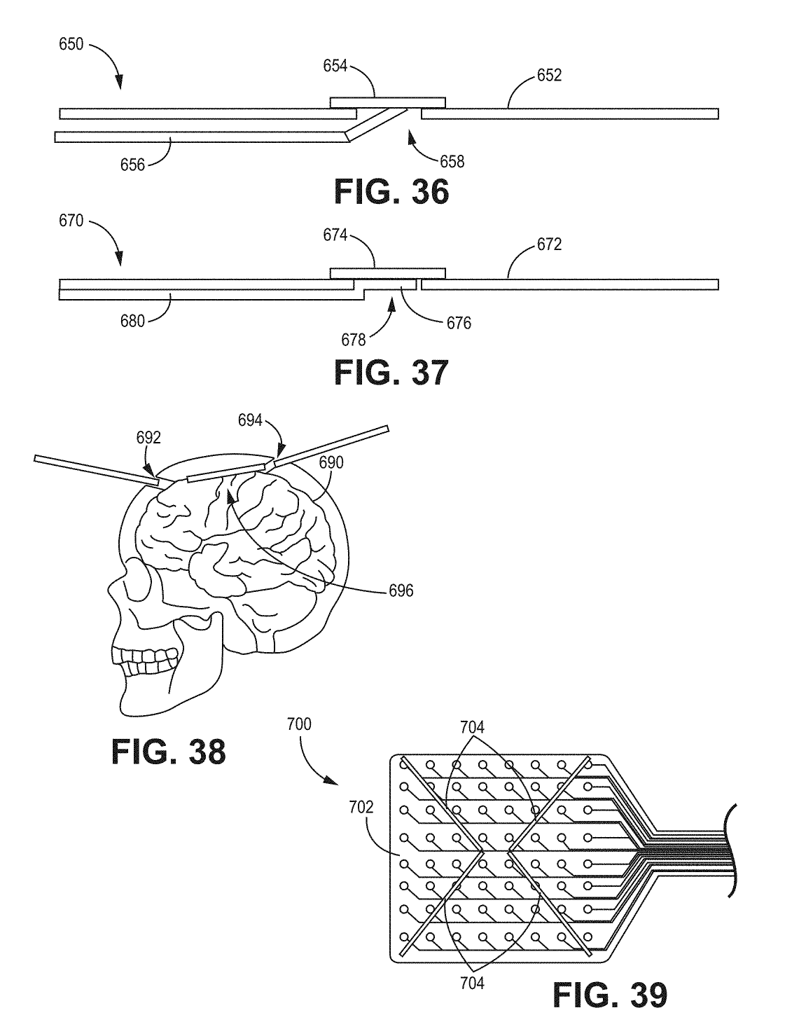

According to certain embodiments, the various electrode arrays, including the thin-film electrode arrays, according to any of the implementations disclosed or contemplated herein, can have a unique arrangement with respect to the positioning of the contacts and the associated contact wires on the array. More specifically, according to one embodiment as shown in FIG. 36, the array 650 can have a pad or base (or “electrode”) 652 with the electrode contact 654 disposed on one side of the pad and the contact wire 656 extending through a hole 658 in the pad 652 and along the side of the pad 652 opposite the contact 654 as shown. In a further embodiment as shown in FIG. 37, the array 670 has a pad 672 with the electrode contact 674 disposed on one side of the pad, a plating 676 disposed in the opening 678, and the contact wire 680 coupled to the plating 676 and being disposed on the side of the pad 672 opposite the contact 674 as shown. In contrast, in known electrode arrays, the contacts and the contact wires (or tails) are positioned on the same side of the base, which results in spatial and connection scheme constraints with respect to the positioning and structure of the contacts and contact wires. In contrast, one result of these unique configurations as set forth herein (and as shown in FIGS. 12 and 13) is that they create a raised profile for the contact—which can result in higher resolution—due to the tail being positioned on the other side of the base 652. That is, the tail being positioned on side opposite the contact means that the contact has a higher height profile in relation to the base 652, thereby resulting in a stronger contact between the contact and the tissue, which results in higher resolution.

A unique minimally-invasive procedure is also contemplated herein. That is, according to one embodiment as depicted in FIG. 38, a new method for performing a procedure of deploying an electrode array into a skull of a patient is provided. It is understood that any of the electrode embodiments disclosed or contemplated herein can be deployed, implanted, or otherwise positioned within the brain tissue of a patient using this minimally-invasive method. In accordance with one specific implementation of this method, two holes (instead of one) 692, 694 are formed in the skull 690 on either side of the target area of the brain tissue. It is understood that the holes are formed using any known medical procedure for forming holes in a patient's skull. Once the two holes 692, 694 are formed, a guidewire (not shown) can be inserted into the skull 690 through one of the two holes and then urged toward and through the second hole such that the guidewire is disposed through both of the holes 692, 694. For example, the guidewire can be inserted into hole 692 and urged distally toward and then out of hole 694. Alternatively, the guidewire can be inserted into hole 694 and urged distally toward and out of hole 692. It is understood that any known guidewire or guidewire in combination with a steerable catheter or other device for inserting and/or positioning a guidewire during a intracranial procedure can be used for this procedure.

Once the guidewire is in place, the electrode array 696 can be inserted into and positioned on the tissue surface as shown. More specifically, according to one specific embodiment, the next step is to insert a catheter or insertion sheath (or the like) (not shown) over the guidewire such that the distal end of the catheter (not shown) is disposed at the desired location for delivering the electrode array 696. In this step, the electrode array 696 can either be previously positioned within the lumen of the catheter (not shown) or it can be inserted through the lumen after the catheter is positioned as desired. In either case, the electrode array 696 can be positioned as desired intracranially through the lumen of the catheter. In certain embodiments in which the distal end of the catheter is positioned at the desired delivery location, the electrode array 696 can be urged distally out of the distal end of the catheter and deployed or otherwise positioned at the desired location. Alternatively, the electrode array 696 can be positioned inside the lumen of the catheter as desired and then the catheter (not shown) can be removed while maintaining the position of the electrode array 696 such that the electrode array 696 is deployed or otherwise positioned at the desired intracranial location. It is understood that the electrode array 696 can be either pushed using a tool positioned proximally of the array 696 or pulled using a tool positioned distally of the array 696. Regardless of the specific steps, once the array 696 is positioned intracranially as desired, the catheter or delivery sheath is removed while the electrode array 696 remains in its desired location. After removal of the delivery sheath, because of the two openings 692, 694 and the fact that delivery tubes or other insertion tools can be used at both openings 692, 694, the electrode array 696 can subsequently be pushed, pulled or otherwise positioned using tools from either opening 692, 694 to adjust or improve the intracranial position of the electrode array 696, thereby making the positioning of the array 696 and refinement thereof easier than is possible with a single opening.

Another electrode array 700 embodiment is depicted in FIG. 39. In this implementation, the array 700 has a pad 702 that has deployable struts 704 attached or otherwise disposed on the pad 702. In one embodiment, the deployable struts 704 are made of a shape-memory material, such as Nitinol or the like. Regardless, the deployable struts 704 are configured to help urge the pad 702 toward a predetermined flat configuration such as the configuration depicted in FIG. 39. Alternatively, the predetermined configuration can have a predetermined curvature as desired. In use, the pad 702 can be inserted into the skull in an undeployed or collapsed configuration such that the pad 702 can fit through a deployment device such as a tube or the like. The tube or other delivery device can help to retain the pad 702 in the undeployed configuration. Once the pad 702 is urged out of the delivery tool at the desired location on the brain tissue surface, the deployable struts 704 begin to urge the pad 702 toward the predetermined flat configuration such that the pad 702 ultimately is deployed to that configuration.

In one embodiment, the struts 704 are disposed on the side of the pad 702 opposite the contact side of the pad 702. The struts 704, according to one implementation, are laminated onto the pad 702. Alternatively, the struts 704 can be attached to the pad 702 via any known process or mechanism.

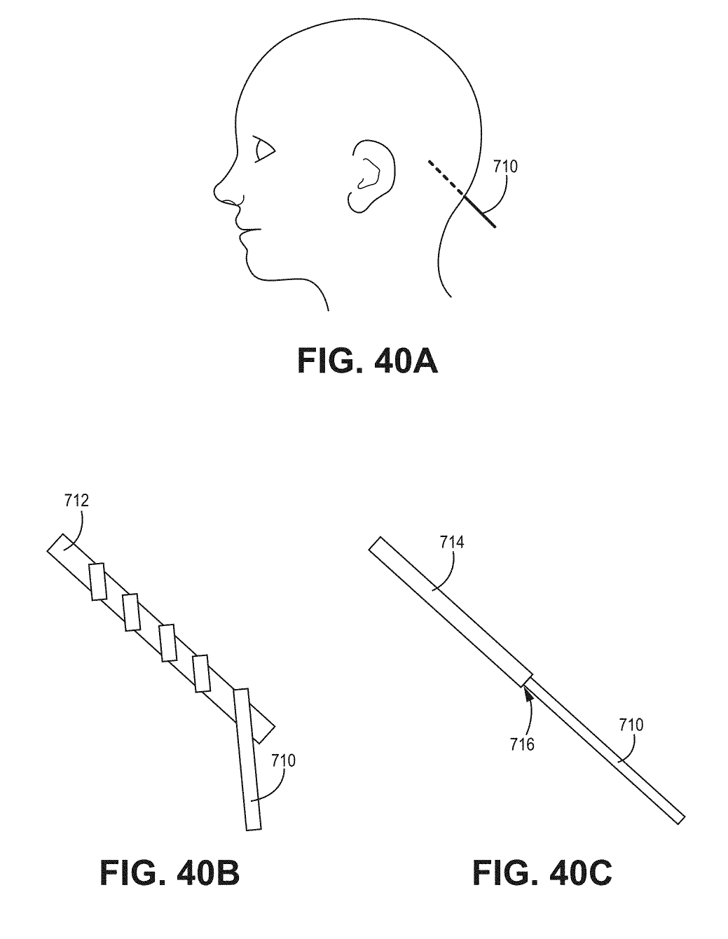

A device, according to one embodiment, is set forth herein that receives and positions the electrode tail 710 for exiting the scalp of the patient as shown in FIG. 40A. It is understood that it is desirable to position the exit point for the electrode tail 710 at the back of the head of the patient. However, while known technologies rely on a tubular-shaped tail (which allows for easy suturing), certain tail embodiments as disclosed herein have a flat cross-section (rather than a round cross-section), which is more difficult to suture. In this embodiment as best shown in FIGS. 40B and 40C, a cannula is provided that can receive the electrode tail 710 and thereby incorporate a tubular structure such that the suturing is simplified. More specifically, in one embodiment as shown in FIG. 40B, the cannula 712 is a solid cannula 712 such that the (flat) tail 710 is wrapped or otherwise positioned around the cannula 712 as shown. Alternatively, the cannula 714 has a lumen 716 such that the tail 710 is positioned through the lumen 716 of the cannula 714. In both implementations, the end result is a tail 710 incorporated or otherwise positioned on or in a tubular structure such that the suturing of the scalp incision is simplified and ensures that the suture can be tightened around a circular structure, thereby reducing or preventing leakage out of the incision.

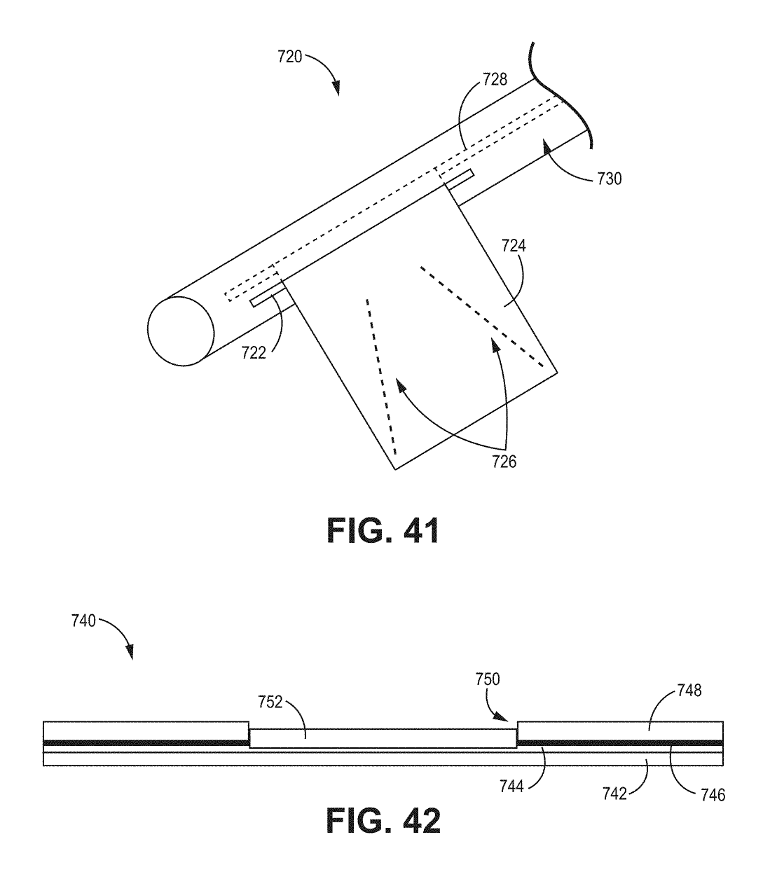

A unique electrode array delivery catheter 720 is depicted in FIG. 41. In this embodiment, the catheter 720 has an opening 722 extending along a length of the catheter 720 near the distal end of the catheter 720. The catheter 720 contains a rotating central rod 728 disposed within the lumen 730 of the catheter 720 that can be rotated (at its proximal end by a user) in relation to the catheter 720. In one embodiment, the rod 728 is a mandrel. The electrode array in this embodiment is a flat pad 724 that can be wrapped or otherwise disposed around the rod 728 such that the pad 724 can be deployed out of the opening 722 by rotating the rod 728 and further can be retracted back into the catheter 720 through the opening 722 by rotating the rod 728 in the other direction. In one embodiment, the pad 724 has a predetermined deployed shape that can be achieved via struts 726 similar to the struts discussed above. Alternatively, the pad 724 can be deployable in a particular configuration according to any known mechanism or process.

In use, according to one embodiment, the catheter 720 remains in place after deployment of the electrode array. In this embodiment, the catheter 720 can also be used as a mandrel for wrapping the electrode tail and as a tunneling structure.

One method of making a thin-film electrode array (and the resulting array 740) is also provided, according to one embodiment as depicted in FIG. 42. Most known thin-film electrodes/arrays are made of copper laminated onto Kapton® or a similar polymeric base component. Typically, the lamination process results in a copper layer that is relatively thick, resulting in a thicker overall electrode array than desired. The unique process contemplated herein relates to a sputtering process, rather than lamination. More specifically, according to one embodiment, the process involves the sputtering of titanium onto the polymeric base (such as Kapton®, for example), instead of copper.

In the specific example depicted in FIG. 42, the base 742 is a 0.00025″ polyimide film base 742, and the sputtering process results in a titanium layer 744 of 200 au. Alternatively, the specific thicknesses can vary according to the thickness of the base 742 and the amount of sputtering that is performed. Because the sputtering process is a process in which the material is added atom-by-atom, the resulting layer of titanium can be much thinner than the laminated copper layer of the known process described above. The next step is to process the resulting base 742 and titanium 744 layers with an etchant, such as, for example, cupric chloride. After the etchant has been applied, the next step, in certain embodiments, is to place a polymeric cover layer 748 on the titanium layer 744. In one implementation, the polymeric cover layer 748 is a polyimide that is attached using an adhesive layer 746 and that is drilled and indexed onto the titanium layer 744. In the specific example depicted in FIG. 42, the polymeric cover layer 748 is a 0.00025″ polyimide film cover layer 748 that is attached to the titanium layer 744 with an adhesive layer 746. According to certain embodiments, gaps or openings are provided or otherwise defined in the cover layer 748 to allow for the contacts 752 to be included therein. At this point in the process, subsequent to the application of the etchant, the titanium layer 744 in the predetermined openings 750 are electroplated with platinum that forms a platinum layer 752 that attaches to the titanium layer 744. In one embodiment, the electroplating process involves placing the exposed titanium layer 744 in an electroplating bath of platinum ions that attach themselves to the titanium layer 744, thereby forming the platinum layer 752 that forms the contacts 752. Alternatively, any known electroplating process can be used.

The resulting electrode array 740 is substantially thinner than the known thin-film arrays created with the known copper lamination process described above. Hence, the resulting device is an ultra-thin film array with biocompatibility that facilitates minimally invasive procedures and provides higher resolution as a result of the advanced, micro-scale manufacturing process.

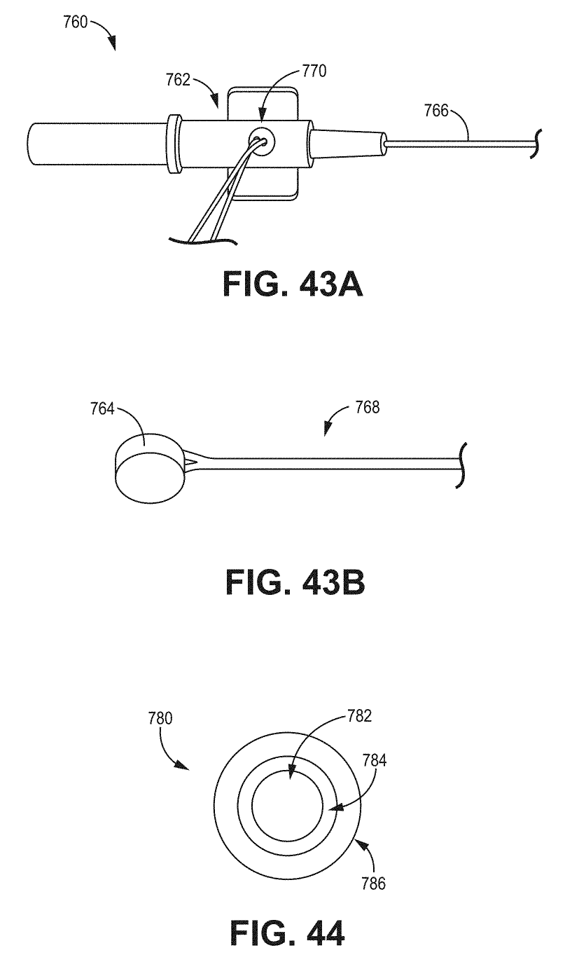

FIGS. 43A and 43B depict a further embodiment of a depth electrode 760 which is substantially similar to the other depth electrode embodiments discussed above—and can have any of the features or components described therein—with the additional feature of a hub 762 (as best shown in FIG. 43A) at its proximal end having a sensor 764 (as best shown in FIG. 43B) disposed therein. Depth electrodes in general typically have a proximal hub disposed at a proximal end of the electrode for coupling thereto and/or handling by a user. In this specific electrode embodiment 760 as shown, the hub 762 has a sensor 764 disposed therein that is coupled to the electrode body 766 that is also disposed therein. As depicted in FIG. 43A, the sensor 764 is disposed within the hub 762 such that the sensor 764 is not visible and the wire(s) 768 extending from the sensor 764 extend out of an opening 770 in the hub 762 such that the sensor 764 can communicate with an external controller and/or speaker (not shown).

In one embodiment, the sensor 764 is a microphone 764 that is coupled to the electrode body 766 such that the microphone 764 can detect sounds through the body 766 that originate from the brain tissue in which the distal end (not shown) of the depth electrode 760 is disposed. The wire(s) 768 can be coupled to a speaker, an oscilloscope, or any other similar known device (not shown) for the user to track the sounds detected by the microphone 764. The speaker (not shown) can reproduce the sounds generated in the brain while the oscilloscope creates a visual representation of those sounds.

According to one implementation, the sensor 764 can be used to track the completion of an ablation procedure. That is, it is believed that the brain tissue being ablated generates a sound when the tissue is sufficiently ablated. It is further believed that the sound is caused by water molecules in the brain tissue that have reached a certain temperature as a result of the ablation such that the water molecules begin to boil, creating a popping or sizzling sound. This sound travels along the length of the electrode body 766 such that the microphone 764 picks up the sound and transmits it along the wire(s) 768 to the output device (speaker, oscilloscope, or the like) (not shown). In use, a user can monitor the output device to determine when to complete an ablation procedure based on the generation of the appropriate sound as described herein.

Another embodiment relates to an improved coated (or plated) wire 780 as shown in FIG. 44 for use in or with any of the various electrode embodiments disclosed or contemplated herein, including, for example, the depth electrodes, cortical electrodes, and other components discussed elsewhere herein. The coated wire 780, according to one embodiment, has a platinum core 782, a gold coating (or “plating” or “layer”) 784 disposed over the core 782, and a platinum coating (or “plating” or “layer”) 786 disposed over the gold coating 784. In known microwires used in neurological devices, the standard material is typically copper, which has both good conductivity and a good signal-to-noise ratio. However, it was discovered that a conductive material with lower conductivity and lower impedance in comparison to copper is desirable in such a wire, because the lower impedance increases the sensitivity of the resulting wire to detect the high-frequency oscillations produced during a seizure of an epileptic patient. For example, platinum is a desirable material, because it has lower conductivity and lower impedance in comparison to copper. More specifically, an outer coating of platinum 786 over an inner coating 784 of a more conductive material such as gold results in a wire 780 that can be used in neurological electrode devices such as any of the device embodiments disclosed or contemplated herein.

Alternatively, the coated wire 780 embodiment can be used in any known medical device requiring transmission of electricity, including, for example, EEG recording devices.

Although the present invention has been described with reference to preferred embodiments, persons skilled in the art will recognize that changes may be made in form and detail without departing from the spirit and scope of the invention.

What is claimed is:

1. A depth electrode comprising:
(a) an elongate, unitary tubular body;
(b) at least one lumen defined through a length of the elongate tubular body; and
(c) at least one electrode array disposed on an outer surface of the elongate tubular body.

2. The depth electrode of claim 1, wherein the at least one electrode array comprises a thin conductive film comprising a plurality of electrode contacts.

3. The depth electrode of claim 1, wherein the at least one electrode array is disposed around the elongate tubular body in a spiral configuration.

4. The depth electrode of claim 1, wherein the at least one electrode array comprises at least two elongate electrode arrays disposed along a length of the elongate tubular body.

5. The depth electrode of claim 1, wherein the at least one electrode array comprises a flat thin conductive film wrapped around at least a portion of the elongate tubular body.

6. The depth electrode of claim 5, wherein the thin conductive film is wrapped around an entire circumference of the elongate tubular body.

7. The depth electrode of claim 1, wherein the at least one lumen is constructed and arranged to allow for passage therethrough of a fluid, particulates, a procedural device, tissue, a treatment composition, or a medication.

8. The depth electrode of claim 1, further comprising at least one electrical component coupled to the at least one electrode array, wherein the at least one electrical component is disposed between the tubular body and the at least one electrode array.

9. The depth electrode of claim 1, further comprising at least one electrical component coupled to the at least one electrode array, wherein the at least one electrical component is disposed at a location external to the tubular body.

10. A positionable cortical electrode comprising:
(a) a thin film pad;
(b) a plurality of electrode contacts disposed in the thin film pad; and
(c) a plurality of flexibility openings defined in the thin film pad, wherein each of the plurality of flexibility openings are constructed and arranged to impart flexibility on the thin film pad.

11. The positionable cortical electrode of claim 10, wherein the thin film pad comprises a first thin film layer and a second thin film layer, wherein the plurality of electrode contacts are disposed between the first and second thin film layers.

12. The positionable cortical electrode of claim 11, further comprising a plurality of contact openings defined in the first thin film layer, wherein one of the plurality of electrode contacts is accessible via one of the plurality of contact openings.

13. The positionable cortical electrode of claim 11, wherein the each of the plurality of flexibility openings are defined in the first and second thin film layers.

14. The positionable cortical electrode of claim 10, wherein the plurality of flexibility openings comprise directional flexibility openings.

15. The positionable cortical electrode of claim 10, wherein the thin film pad comprises a rounded edge.

16. A method of implanting an intracranial electrode array, the method comprising:
forming first and second holes in a skull of a patient;
inserting a guidewire through the first hole;
urging the guidewire distally toward and through the second hole such that the guidewire is disposed through the first and second holes;
urging an introduction sheath distally over the guidewire to a target intracranial position;
positioning the intracranial electrode array at the target intracranial position via the introduction sheath; and
removing the introduction sheath and the guidewire.

17. The method of claim 16, further comprising adjusting a final position of the intracranial electrode array.

18. The method of claim 17, wherein the adjusting the final position of the intracranial array comprises using a tool disposed through the first or second hole.

19. The method of claim 16, wherein the positioning the intracranial electrode array at the target intracranial position via the introduction sheath further comprises positioning the intracranial electrode array in the introduction sheath prior to urging the introduction sheath distally to the target intracranial position.

20. The method of claim 16, wherein the positioning the intracranial electrode array at the target intracranial position via the introduction sheath further comprises urging the intracranial electrode array into and through the introduction sheath after urging the introduction sheath distally to the target intracranial position.
